(19) United States
(12) Patent Application Publication
(10) Pub. No.: US 2016/0199288 A1
(43) Pub. Date: Jul. 14, 2016

(54) COSMETIC ANTIPERSPIRANTS WITH AMIDOSULPHONIC ACIDS

(71) Applicant: Henkel AG & Co. KGaA, Duesseldorf (DE)

(72) Inventors: Bernhard Banowski, Duesseldorf (DE); Detlef Keller, Mettmann (DE)

(73) Assignee: Henkel AG & Co. KGaA, Duessledorf (DE)

(21) Appl. No.: 15/080,012

(22) Filed: Mar. 24, 2016

Related U.S. Application Data
(63) Continuation of application No. PCT/DE2014/200512, filed on Sep. 26, 2014.

(30) Foreign Application Priority Data
Oct. 15, 2013 (DE) 10 2013 220 786.7

(51) Int. Cl. A61K 8/92; A61K 8/46; A61K 8/28; A61K 8/49; A61Q 15/00; A61K 8/26

(52) U.S. Cl. CPC A61K 8/92; A61Q 15/00; A61K 8/26; A61K 8/28; A61K 8/49; A61K 8/466

(57) ABSTRACT

The present invention relates to a cosmetic antiperspirant agent including a) at least one substance selected from the group of cosmetic oils which are liquid at 20° C. and 1013 hPa, odorants, and waxes, b) at least one antiperspirant aluminum salt in a total quantity of 1 to 80% by weight, based on the total weight of the cosmetic antiperspirant agent, and c) at least one compound of formula (M-I)

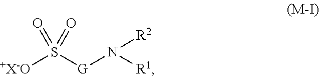

### FIELD OF THE INVENTION

The present invention generally relates to a cosmetic antiperspirant agent having at least one antiperspirant aluminum salt and at least one sulfonic acid functionalized with a nitrogen group, and wherein the addition of the at least one functionalized sulfonic acid results in effective activation of the at least one antiperspirant aluminum salt and in stabilization of this activated state.

Moreover, the present invention relates to the use of a combination of at least one antiperspirant aluminum salt and at least one sulfonic acid functionalized with a nitrogen group for reducing and/or preventing underarm perspiration.

Furthermore, the present invention relates to a nontherapeutic cosmetic method for preventing and/or reducing the perspiration of the body, in which the antiperspirant agent according to the invention is applied to the skin, in particular to the skin of the armpits.

Lastly, the present invention relates to the use of a sulfonic acid functionalized with a nitrogen group for activating and/or stabilizing an antiperspirant aluminum salt.

### BACKGROUND OF THE INVENTION

Washing, cleaning, and caring for the body is a basic human need, and modern industry continually strives to meet this need in various ways. The continual removal, or at least reduction, of body odor and underarm wetness is particularly important for daily hygiene. Numerous specialized deodorizing or antiperspirant personal care products are known in the prior art which have been developed for use in body regions having a high density of sweat glands, in particular in the armpit area. These products are provided in various administration forms, for example as a powder, in stick form, as an aerosol spray, pump spray, liquid and gel-like roll-on application, cream, gel, and as impregnated flexible substrates (deodorant wipes).

Cosmetic antiperspirants of the prior art contain, in addition to at least one oil or a fatty substance and an odorant component or fragrance, at least one antiperspirant salt.

Typically basic aluminum and aluminum-zirconium halides in the form of chlorides are used as the antiperspirant salt, since, unlike the nonbasic aluminum and aluminum-zirconium chlorides, they do not have a skin-irritating effect. However, the disadvantage of the basic aluminum and aluminum-zirconium halides lies in the formation of higher-molecular oligomeric and polymeric aluminum species which significantly reduce the activity of the basic aluminum and aluminum-zirconium salts in antiperspirants.

For this reason, attempts have been made in the prior art to increase the activity of the basic aluminum and aluminum-zirconium halides by activation, while at the same time maintaining good skin tolerance. Thus, basic aluminum and aluminum-zirconium halides, in particular aluminum chlorides, which have been obtained by thermal treatment are described in the publications EP 0308937 A2, EP 0183171 A2, U.S. Pat. No. 4,359,456 A, and EP 0191628 A2. In gel permeation chromatography (GPC), the heat-treated, activated basic aluminum and aluminum-zirconium halides have a lower proportion of high-molecular species compared to untreated basic aluminum and aluminum-zirconium halides, and thus have increased activity against sweat secretion in antiperspirants.

In addition, the activity of basic aluminum and aluminum-zirconium halides may be increased by incorporating organic acids as complexing ligands. Thus, the publications U.S. Pat. No. 3,542,919 A, U.S. Pat. No. 3,553,316 A, U.S. Pat. No. 3,991,176 A, and WO 2005/092795 A1 disclose methods for producing such stabilized aluminum and aluminum-zirconium halides which have a higher proportion of short-chain species and are present in activated form.

One disadvantage of the above-mentioned activated basic aluminum and aluminum-zirconium halides of the prior art is that the antiperspirant effect decreases with increasing storage time and/or when protic solvents are used. This is due to the fact that the polymer distribution of the aluminum species is based on a reversible equilibrium, and in protic solvents this equilibrium shifts over time to higher-molecular, and thus less effective, aluminum species.

Therefore, there is a need for cosmetic antiperspirant agents which have no shift of the polymer distribution to long-chain polymers, and thus, no significant decrease in the antiperspirant effect, even over long storage periods and/or when large quantities of protic solvents are used. A further aim is for the antiperspirants to be economically manufacturable.

The object of the present invention is to provide a cosmetic antiperspirant agent which avoids or at least mitigates the disadvantages of the prior art, and for which no significant decrease in the activation of antiperspirant aluminum salts occurs during extended storage periods and/or in the presence of protic solvents.

It has now surprisingly been found that the use of particular sulfonic acids functionalized with nitrogen-containing groups or the salts thereof in cosmetic agents including antiperspirant aluminum salts results in significantly increased formation of short-chain aluminum species, and in effective stabilization of these short-chain aluminum species, even over long storage periods.

Furthermore, other desirable features and characteristics of the present invention will become apparent from the subsequent detailed description of the invention and the appended claims, taken in conjunction with this background of the invention.

### BRIEF SUMMARY OF THE INVENTION

A cosmetic antiperspirant agent including at least one substance selected from the group of cosmetic oils which are liquid at 20° C. and 1013 hPa, odorants, and waxes; at least one antiperspirant aluminum salt in a total quantity of 1 to 80% by weight, based on the total weight of the cosmetic antiperspirant agent; and at least one compound of formula (M-I)

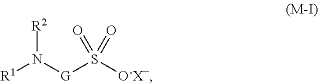

where G stands for a chemical bond or a linear or branched alkylene group having 1 to 6 carbon atoms; R¹ and R² independently stand for hydrogen or a linear or branched, saturated or unsaturated alkylene group having 1 to 10 carbon atoms, or R¹ and R² form a cyclic group having 5 to 7 carbon atoms and at least one heteroatom selected from the group of N, O, and S; and X⁺ stands for H⁺, Li⁺, Na⁺, K⁺, NH₄⁻, ½Mg²⁺, ½Ca²⁺, ½Mn²⁻, ½Zn²⁺, ⅓Al³⁺, ¼Zr⁴⁺, or at least one antiperspirant aluminum salt.

Use of a combination of at least one substance selected from the group of cosmetic oils which are liquid at 20° C. and 1013 hPa, odorants, and waxes, at least one antiperspirant aluminum salt in a total quantity of 1 to 80% by weight, based on the total weight of the combination, and at least one compound of formula (M-I)

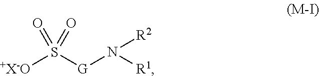

where G stands for a direct bond or a linear or branched alkylene group having 1 to 6 carbon atoms; R¹ and R² independently stand for hydrogen or a linear or branched, saturated or unsaturated alkylene group having 1 to 10 carbon atoms, or R¹ and R² form a cyclic group having 5 to 7 carbon atoms and at least one heteroatom selected from the group of N, O, and S; and X⁺ stands for H⁺, Li⁺, Na⁺, K⁺, NH₄⁻, ½Mg²⁺, ½Ca²⁺, ½Mn²⁻, ½Zn²⁺, ⅓Al⁺, ¼Zr⁴⁺, or at least one antiperspirant aluminum salt, for reducing and/or preventing perspiration.

Use of at least one compound of formula (M-I)

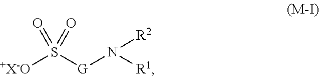

where G stands for a direct bond or a linear or branched alkylene group having 1 to 6 carbon atoms; R¹ and R² independently stand for hydrogen or a linear or branched, saturated or unsaturated alkylene group having 1 to 10 carbon atoms, or R¹ and R² form a cyclic group having 5 to 7 carbon atoms and at least one heteroatom selected from the group of N, O, and S; and X⁺ stands for H⁺, Li⁺, Na⁺, K⁺, NH₄⁻, ½Mg²⁺, ½Ca²⁺, ½Mn²⁻, ½Zn²⁺, ⅓Al³⁺¼Zr⁴⁺, or at least one antiperspirant aluminum salt, for activating and/or stabilizing at least one antiperspirant aluminum salt.

A nontherapeutic cosmetic method for preventing and/or reducing the perspiration of the body, in which a cosmetic antiperspirant agent including at least one substance selected from the group of cosmetic oils which are liquid at 20° C. and 1013 hPa, odorants, and waxes, at least one antiperspirant aluminum salt in a total quantity of 1 to 80% by weight, based on the total weight of the cosmetic antiperspirant agent, and at least one compound of formula (M-I) is applied to the skin, in particular to the skin of the armpits.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The subject matter of the present invention is a cosmetic antiperspirant agent including
a) at least one substance selected from the group of cosmetic oils which are liquid at 20° C. and 1013 hPa, odorants, and waxes,
b) at least one antiperspirant aluminum salt in a total quantity of 1 to 80% by weight, based on the total weight of the cosmetic antiperspirant agent, and
c) at least one compound of formula (M-I)

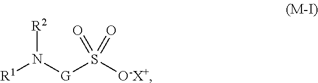

where
G stands for a chemical bond or a linear or branched alkylene group having 1 to 6 carbon atoms,
R¹ and R² independently stand for hydrogen or a linear or branched, saturated or unsaturated alkylene group having 1 to 10 carbon atoms, or R¹ and R² form a cyclic group having 5 to 7 carbon atoms and at least one heteroatom selected from the group of N, O, and S, and
X⁺ stands for H⁻, Li⁺, Na⁺, K⁺, NH₄⁺, ½Mg²⁺, ½Ca²⁺, ½Mn²⁺, ½Zn²⁺, ⅓Al³⁻, ¼Zr⁴⁺, or at least one antiperspirant aluminum salt.

A significant increase in the proportion of short-chain species of the antiperspirant aluminum salts is achieved by combining antiperspirant aluminum salts with particular sulfonic acids of formula (M-I), without being restricted to this theory. Furthermore, the short-chain species formed in this way are very well stabilized by the above-mentioned sulfonic acids, thus ensuring an improved antiperspirant effect over a long period of time. By use of the particular sulfonic acids, in particular antiperspirants having a high proportion of protic solvents may be produced which, despite the large quantities of protic solvents, have an excellent antiperspirant effect even over extended storage periods.

According to the invention, the term “antiperspirant” is understood to mean the decrease or reduction in the transpiration of the sweat glands of the body.

In addition, within the meaning of the present invention the term “cosmetic oil” is understood to mean an oil which is suitable for cosmetic use and which is not miscible with water. Moreover, the cosmetic oil used according to the present invention does not involve either odorants or essential oils.

In addition, within the meaning of the present invention the term “odorants” is understood to mean substances having a molar mass of 74 to 300 g/mol which contain at least one osmophoric group in the molecule and which have an odor and/or taste, i.e., which are capable of stimulating the receptors of the hair cells of the olfactory system. Osmophoric groups, in the form of hydroxy groups, formyl groups, oxo groups, alkoxycarbonyl groups, nitrile groups, nitro groups, azide groups, etc., are groups which are covalently bonded to the molecular structure. In this regard, within the meaning of the present invention, liquid fragrance oils, fragrances, or fragrance oil components, also at 20° C. and 1013 hPa, fall under the term “odorants.”

Furthermore, within the scope of the present invention the term “waxes” is understood to mean substances which are kneadable at 20° C. or which have solid to brittle hardness, which have a rough to fine crystalline structure, and which have colored translucence to opaqueness but are not transparent. Moreover, these substances melt above 25° C. without decomposition, are slightly liquid (less viscous) just above the melting point, have a strongly temperature-dependent consistency and solubility, and are polishable under light pressure.

According to the invention, the term “volatile cosmetic oil” refers to cosmetic oils having a vapor pressure of 2.66 Pa to 40,000 Pa (0.02 to 300 mm Hg), preferably 10 to 12,000 Pa (0.1 to 90 mm Hg), more preferably 13 to 3000 Pa (0.1 to 23 mm Hg), in particular 15 to 500 Pa (0.1 to 4 mm Hg), at 20° C. and an ambient pressure of 1013 hPa.

Furthermore, within the meaning of the present invention the term “nonvolatile cosmetic oils” is understood to mean cosmetic oils having a vapor pressure of less than 2.66 Pa (0.02 mm Hg) at 20° C. and an ambient pressure of 1013 hPa.

Moreover, the term “fatty acid” as used within the scope of the present invention is understood to mean aliphatic carboxylic acids which have unbranched or branched hydrocarbon moieties having 4 to 40 carbon atoms. The fatty acids used within the scope of the present invention may be naturally occurring as well as synthetically produced fatty acids. In addition, the fatty acids may be singly or multiply unsaturated.

Lastly, within the scope of the present invention the term “fatty alcohol” is understood to mean aliphatic, monohydric primary alcohols which have unbranched or branched hydrocarbon moieties having 4 to 40 carbon atoms. The fatty alcohols used within the scope of the invention may also be singly or multiply unsaturated.

In the present context, unless stated otherwise, the indication “% by weight” refers to the total weight of cosmetic antiperspirant agents according to the invention, not including any propellant which may be present.

According to one preferred embodiment of the present invention, the cosmetic oil which is liquid at 20° C. and 1013 hPa is selected from the group of
(i) volatile cyclic silicone oils, in particular cyclotrisiloxane, cyclotetrasiloxane, cyclopentasiloxane, and cyclohexasiloxane, and linear silicone oils having 2 to 10 siloxane units, in particular hexamethyldisiloxane, octamethyltrisiloxane, decamethyltetrasiloxane;
(ii) volatile nonsilicone oils, in particular liquid paraffin oils and isoparaffin oils, such as isodecane, isoundecane, isododecane, isotridecane, isotetradecane, isopentadecane, isohexadecane, and isoeicosane;
(iii) nonvolatile silicone oils, in particular higher molecular linear polyalkylsiloxanes;
(iv) nonvolatile nonsilicone oils, in particular the esters of linear or branched, saturated or unsaturated C₂₋₃₀ fatty alcohols with linear or branched, saturated or unsaturated C₂₋₃₀ fatty acids, which may be hydroxylated, the C₈-C₂₂ fatty alcohol esters of monovalent or polyvalent C₂-C₇ hydroxycarboxylic acids, the triethyl citrates, the branched saturated or unsaturated C₆₋₃₀ fatty alcohols, the mono-, di-, and triglycerides of linear or branched, saturated or unsaturated, optionally hydroxylated C₈₋₃₀ fatty acids, the dicarboxylic acid esters of linear or branched C₂-C₁₀ alkanols, the addition products of ethylene oxide and/or propylene oxide with monohydric or polyhydric C₃₋₂₂ alkanols, which may optionally be esterified, the symmetrical, asymmetrical, or cyclic esters of carbonic acid with fatty alcohols, the esters of dimers of unsaturated C₁₂₋₂₂ fatty acids with monohydric linear, branched, and cyclic C₂₋₁₈ alkanols or C₂₋₆ alkanols, the benzoic acid esters of linear or branched C₈₋₂₂ alkanols, such as benzoic acid C₁₂₋₁₅ alkyl esters and benzoic acid isostearyl ester and benzoic acid octyldodecyl ester, the synthetic hydrocarbons such as polyisobutene and polydecene, the alicyclic hydrocarbons; and
(v) the mixtures thereof.

The use of volatile silicone oils and volatile nonsilicone oils in the cosmetic antiperspirant agents according to the invention results in a drier feeling on the skin and more rapid release of the antiperspirant aluminum salt.

The cyclic volatile silicone oils which are usable within the scope of the invention have a vapor pressure of 13 to 15 Pa (0.1 mm Hg) at 20° C. and an ambient pressure of 1013 hPa. Furthermore, a low-molecular phenyl trimethicone having a vapor pressure of approximately 2000 Pa (15 mm Hg) at 20° C. and an ambient pressure of 1013 hPa may also be used according to the invention as a linear volatile silicone oil. Due to the high persistence of cyclodimethicones in the environment, however, it may be preferred according to the invention to use 0 to less than 1% by weight, preferably 0 to less than 0.1% by weight, of cyclic volatile silicone oils in the cosmetic antiperspirant agents according to the invention.

According to the invention, volatile nonsilicone oils in the form of C₁₀-13 isoparaffin mixtures having a vapor pressure of 10 to 400 Pa (0.08 to 3 mm Hg), preferably 13 to 100 Pa (0.1 to 0.8 mm Hg), at 20° C. and an ambient pressure of 1013 hPa are preferably used. Within the scope of the present invention it is preferred when the volatile C₈-C₁₆ isoparaffin is contained in a total quantity of 1 to 60% by weight, preferably 3 to 45% by weight, more preferably 5 to 40% by weight, in particular 8 to 20% by weight, based on the total weight of the cosmetic antiperspirant agent. Of course, it is likewise possible to formulate cosmetic antiperspirant agents according to the invention with a small proportion of volatile oils, i.e., with 0.5 to 15% by weight of volatile oils, based on the total weight of the cosmetic antiperspirant agent, or even with no volatile oils at all.

For masking of insoluble components such as talc or antiperspirant aluminum salts dried on the skin, it may be preferred according to the invention for the cosmetic antiperspirant agents to contain a nonvolatile silicone oil and/or a nonvolatile nonsilicone oil.

In this regard, cosmetic antiperspirant agents preferred according to the invention contain at least one ester of linear or branched, saturated or unsaturated fatty alcohols having 2 to 30 carbon atoms with linear or branched, saturated or unsaturated fatty acids having 2 to 30 carbon atoms, which may be hydroxylated, in a total quantity of 1 to 30% by weight, preferably 5 to 26% by weight, more preferably 9 to 24% by weight, in particular 12 to 17% by weight, based on the total weight of the cosmetic antiperspirant agent.

Within the scope of the present invention, linear polyalkylsiloxanes having a kinematic viscosity of 5 to 2000 cSt at 25° C., in particular linear polydimethylsiloxanes having a kinematic viscosity of 5 to 2000 cSt, preferably 10 to 350 cSt, in particular 50 to 100 cSt, at 25° C. may be used as nonvolatile silicone oils. The above-mentioned nonvolatile silicone oils are available under the trade names Dow Corning 200 from Dow Corning and Xiameter PMX from Xiameter. Further preferred nonvolatile silicone oils are phenyl trimethicones having a kinematic viscosity of 10 to 100 cSt, preferably 15 to 30 cSt, at 25° C., and cetyl dimethicones.

According to the invention, the use of mixtures of the above-mentioned cosmetic oils, in particular nonvolatile and volatile cosmetic oils, is also preferred, since in this way parameters such as feeling on the skin, visibility of residue, and stability of the cosmetic antiperspirant agent according to the invention may be set, and the agent may thus be better adapted to the needs of the consumer.

Within the scope of the present invention, it is preferred when the cosmetic oil which is liquid at 20° C. and 1013 hPa is contained in a total quantity of 1 to 95% by weight, preferably 10 to 85% by weight, more preferably 20 to 75% by weight, particularly preferably 35 to 70% by weight, in particular 50 to 60% by weight, based on the total weight of the cosmetic antiperspirant agent.

However, it may also be preferred when the cosmetic oil which is liquid at 20° C. and 1013 hPa is contained in a total quantity of 0.2 to 70% by weight, preferably 2 to 60% by weight, more preferably 3 to 50% by weight, particularly preferably 5 to 35% by weight, in particular 8 to 20% by weight, based on the total weight of the cosmetic antiperspirant agent. In this regard, it may also be provided according to the invention for the antiperspirant agents according to the invention to contain less than 0.2% by weight, preferably less than 0.1% by weight, in particular 0% by weight, of the cosmetic oil which is liquid at 20° C. and 1013 hPa. The use of only extremely small quantities of the cosmetic oil which is liquid at 20° C. and 1013 hPa is preferred in particular for cosmetic antiperspirant agents according to the invention which are present in aqueous, aqueous-alcoholic, or aqueous-glycolic form.

According to one preferred embodiment of the present invention, the odorant is selected from the group of
(i) esters, in particular benzyl acetate, phenoxyethyl isobutyrate, p-tert-butylcyclohexyl acetate, linalyl acetate, dimethylbenzylcarbinyl acetate (DMBCA), phenylethyl acetate, ethylmethylphenyl glycinate, allylcyclohexyl propionate, styrallyl propionate, benzyl salicylate, cyclohexyl salicylate, Floramat, Melusat, and jasmecyclate;
(ii) ethers, in particular benzyl ethyl ether and ambroxan;
(iii) aldehydes, in particular linear alkanals having 8 to 18 carbon atoms, citral, citronellal, citronellyloxy acetaldehyde, cyclamen aldehyde, Lilial, and bourgeonal;
(iv) ketones, in particular ionones, alpha-isomethylionone, and methyl cedryl ketone;
(v) alcohols, in particular anethol, citronellol, eugenol, geraniol, linalool, phenylethyl alcohol, and terpineol;
(vi) hydrocarbons, in particular terpenes such as limonene and pinene; and
(vii) the mixtures thereof.

However, it is preferred to use mixtures of various odorants which together produce a pleasing fragrance.

Cosmetic antiperspirant agents according to the invention having a particularly pleasing fragrance are obtained when the odorant is contained in a total quantity of 0.00001 to 10% by weight, preferably 0.001 to 9% by weight, more preferably 0.01 to 8% by weight, particularly preferably 0.5 to 7% by weight, in particular 1 to 6% by weight, based on the total weight of the cosmetic antiperspirant agent. In this regard, however, it may also be provided for the odorant to be contained in a total quantity of 0.00001 to 5% by weight, preferably 0.001 to 4% by weight, more preferably 0.01 to 3% by weight, particularly preferably 0.1 to 2% by weight, in particular 0.2 to 1.5% by weight, based on the total weight of the propellant-containing cosmetic antiperspirant agent.

According to another preferred embodiment of the present invention, the wax is selected from the group of
(i) fatty acid glycerin mono-, di-, and triesters;
(ii) Butyrospermum parkii (shea butter);
(iii) esters of saturated monohydric C₈₋₁₈ alcohols with saturated C₁₂₋₁₈ monocarboxylic acids;
(iv) linear primary C₁₂-C₂₄ alkanols;
(v) esters of a saturated monohydric C₁₆₋₆₀ alkanol and a saturated C₈-C₃₆ monocarboxylic acid, in particular cetyl behenate, stearyl behenate, and C₂₀-C₄₀ alkyl stearate;
(vi) glycerin triesters of saturated linear C₁₂-C₃₀ carboxylic acids, which may be hydroxylated, in particular hydrogenated palm oil, hydrogenated coconut oil, hydrogenated castor oil, glyceryl tribehenate. and glyceryl tri-12-hydroxystearate;
(vii) natural plant waxes, in particular candelilla wax, carnauba wax, Japan wax, sugar cane wax, ouricouri wax, cork wax, sunflower wax, fruit waxes;
(viii) animal waxes, in particular beeswax, shellac wax, and spermaceti;
(ix) synthetic waxes, in particular montan ester waxes, hydrogenated jojoba waxes, and sasol waxes, polyalkylene waxes and polyethylene glycol waxes, C₂₀-C₄₀ dialkyl esters of dimer acids, C₃₀₋₅₀ alkyl beeswax, and alkyl and alkylaryl esters of dimer fatty acids, paraffin waxes; and
(x) the mixtures thereof.

Particularly preferred are commercial products with the INCI name Cocoglycerides, in particular the commercial products Novata® (from BASF), particularly preferably Novata® AB, a mixture of C₁₂₋₁₈ mono-, di-, and triglycerides which melts in the range of 30 to 32° C., and the products of the Softisan line (Sasol Germany GmbH) with the INCI name Hydrogenated Cocoglycerides, in particular Softisan 100, 133, 134, 138, 142. Additional preferred esters of saturated monohydric C₁₂₋₁₈ alcohols with saturated C₁₂₋₁₈ monocarboxylic acids are stearyl laurate, cetearyl stearate (Crodamol® CSS, for example), cetyl palmitate (Cutina® CP, for example), and myristyl myristate (Cetiol® MM, for example). In addition, a C₂₀-C₄₀ alkyl stearate is preferably used as a wax component. This ester is known under the names Kesterwachs® K82H or Kesterwachs® K80H, and is marketed by Koster Keunen Inc.

Within the scope of the present invention, it is preferred for the wax to be contained in a total quantity of 0.01 to 20% by weight, preferably 3 to 20% by weight, more preferably 5 to 18% by weight, in particular 6 to 15% by weight, based on the total weight of the cosmetic antiperspirant agent.

Within the scope of the present invention, a particularly good antiperspirant effect is obtained when the antiperspirant aluminum salt is contained in a total quantity of 2 to 40% by weight, preferably 3 to 35% by weight, more preferably 4 to 32% by weight, particularly preferably 5 to 28% by weight, even more preferably 8 to 25% by weight, in particular 12 to 22% by weight, based on the total weight of the cosmetic antiperspirant agent. However, it may also be provided that the antiperspirant aluminum salt is contained in a total quantity of 0.1 to 20% by weight, preferably 0.5 to 15% by weight, more preferably 1 to 12% by weight, even more preferably 1.5 to 10% by weight, in particular 2 to 8% by weight, based on the total weight of the propellant-containing antiperspirant composition.

Within the scope of the present invention, the antiperspirant aluminum salt may be selected from the group of
(i) water-soluble astringent inorganic salts of aluminum, in particular aluminum chlorohydrate, aluminum sesquichlorohydrate, aluminum dichlorohydrate, aluminum hydroxide, potassium aluminum sulfate, aluminum bromohydrate, aluminum chloride, aluminum sulfate;
(ii) water-soluble astringent organic salts of aluminum, in particular aluminum chlorohydrex propylene glycol, aluminum chlorohydrex polyethylene glycol, aluminum-propylene glycol complexes, aluminum sesquichlorohydrex propylene glycol, aluminum sesquichlorohydrex polyethylene glycol, aluminum dichlorohydrex propylene glycol, aluminum dichlorohydrex polyethylene glycol, aluminum undecylenoyl collagen amino acid, sodium aluminum lactate, sodium aluminum chlorohydroxy lactate, aluminum lipoamino acids, aluminum lactate, aluminum chlorohydroxy allantoinate, sodium aluminum chlorohydroxy lactate;
(iii) water-soluble astringent inorganic aluminum-zirconium salts, in particular aluminum zirconium trichlorohydrate, aluminum zirconium tetrachlorohydrate, aluminum zirconium pentachlorohydrate, aluminum zirconium octachlorohydrate;
(iv) water-soluble astringent organic aluminum-zirconium salts, in particular aluminum-zirconium/propylene glycol complexes, aluminum zirconium trichlorohydrex glycine, aluminum zirconium tetrachlorohydrex glycine, aluminum zirconium pentachlorohydrex glycine, aluminum zirconium octachlorohydrex glycine; and
(v) the mixtures thereof.

According to the invention, the term “antiperspirant aluminum salts” is not understood to mean aluminosilicates and zeolites. In addition, according to the invention, water-soluble aluminum salts are understood to mean those salts which have a solubility of at least 3% by weight at 20° C.; i.e., at least 3 g of the antiperspirant aluminum salt dissolves in 97 g water at 20° C.

Particularly preferred inorganic aluminum salts are selected from aluminum chlorohydrate, in particular aluminum chlorohydrate of the general formula [Al₂(OH)₅Cl.1-6H₂O]ₙ, preferably [Al₂(OH)₅Cl₂.2-3H₂O]ₙ, which may be present in unactivated (polymerized) or in activated (depolymerized) form, and aluminum chlorohydrate with the general formula [Al₂(OH)₄Cl₂.1-6H₂O]ₙ, preferably [Al₂(OH)₄Cl₂.2-3H₂O]ₙ, which may be present in unactivated (polymerized) or in activated (depolymerized) form. The production of these types of antiperspirant aluminum salts is disclosed in the publications U.S. Pat. No. 3,887,692 A, U.S. Pat. No. 3,904,741 A, U.S. Pat. No. 4,359,456 A, GB 2048229 A, and GB 1347950 A, for example.

According to the invention, particularly preferred antiperspirant aluminum salts are selected from so-called “activated” aluminum salts, also referred to as antiperspirant active substances “with enhanced activity.” These types of active substances are known in the prior art and are also commercially available. Production thereof is disclosed in the publications GB 2048229 A, U.S. Pat. No. 4,775,528 A, and U.S. Pat. No. 6,010,688 A, for example. Activated aluminum salts are generally produced by heat treatment of a diluted solution of the corresponding salt (for example, a solution including 10% by weight salt) in order to increase its HPLC peak 4-to-peak 3 area ratio. The activated salt may subsequently be dried, in particular spray-dried, to form a powder. Besides spray drying, roller drying, for example, is also suitable. Activated aluminum salts typically have an HPLC peak 4-to-peak 3 area ratio of at least 0.4, preferably at least 0.7, in particular at least 0.9, whereby at least 70% of the aluminum is to be assigned to these HPLC peaks.

In this regard, “activated” aluminum-zirconium salts are also known which have a high HPLC peak 5 aluminum content, in particular a peak 5 area of at least 33%, preferably at least 45%, based on the entire area under peaks 2 to 5, measured with HPLC, of a 10% by weight aqueous solution of the active substance under conditions in which the aluminum species are resolved in at least 4 successive peaks (referred to as peaks 2 to 5). Preferred aluminum-zirconium salts having a high HPLC peak 5 aluminum content (also referred to as “E⁵AZCH”) are disclosed in the publications U.S. Pat. No. 6,436,381 A and U.S. Pat. No. 6,649,152 A, for example. Furthermore, the above-mentioned activated aluminum-zirconium salt may be additionally stabilized with a water-soluble strontium salt and/or with a water-soluble calcium salt, as disclosed in the publication U.S. Pat. No. 6,923,952 A, for example.

It is likewise possible according to the invention to use antiperspirant aluminum salts as nonaqueous solutions or solublizates of an activated antiperspirant aluminum or aluminum-zirconium salt, for example according to the publication U.S. Pat. No. 6,010,688 A. Such aluminum or aluminum-zirconium salts are stabilized against loss of activation of the salt by adding an effective quantity of a polyhydric alcohol having 3 to 6 carbon atoms and 3 to 6 hydroxyl groups, preferably propylene glycol, sorbitol, and pentaerythrite.

Also particularly preferred are complexes of activated antiperspirant aluminum or aluminum-zirconium salts with a polyhydric alcohol having 20 to 50% by weight, preferably 20 to 42% by weight, of activated antiperspirant aluminum or aluminum-zirconium salt and 2 to 16% by weight of molecularly bound water, with the remaining up to 100% by weight being at least one polyhydric alcohol having 3 to 6 carbon atoms and 3 to 6 hydroxyl groups. Propylene glycol, propylene glycol/sorbitol mixtures, and propylene glycol/pentaerythrite mixtures are preferred alcohols of this type. Such complexes of an activated antiperspirant aluminum or aluminum-zirconium salt having a polyhydric alcohol, preferred according to the invention, are disclosed in the publications U.S. Pat. No. 5,643,558 A and U.S. Pat. No. 6,245,325 A, for example.

Within the scope of the present invention, it is likewise possible to use basic calcium-aluminum salts, as disclosed in the publication U.S. Pat. No. 2,571,030 A, for example, as antiperspirant aluminum salts. These salts may be obtained by reacting calcium carbonate with aluminum chlorohydroxide or aluminum chloride and aluminum powder, or by adding calcium chloride dihydrate to aluminum chlorohydroxide. However, it is also possible to use aluminum-zirconium complexes that are buffered with salts of amino acids, in particular with alkali and alkaline earth glycinates, as disclosed in the publication U.S. Pat. No. 4,017,599 A, for example.

The aluminum or aluminum-zirconium salts stated in the publications U.S. Pat. No. 6,245,325 A, U.S. Pat. No. 6,042,816 A, U.S. Pat. No. 6,245,325 A, U.S. Pat. No. 6,042,816 A, U.S. Pat. No. 6,245,325 A, U.S. Pat. No. 6,042,816 A, U.S. Pat. No. 6,245,325 A, U.S. Pat. No. 6,042,816 A or U.S. Pat. No. 7,105,691 A may be used as antiperspirant activated aluminum and aluminum-zirconium salts preferred according to the invention, which are preferably stabilized by amino acids, in particular glycine, hydroxyalkanoic acids, in particular glycolic acid and lactic acid, or betaines.

Further preferred activated aluminum salts are those of general formula Al₂(OH)₆₋ₐXa, where X stands for Cl, Br, I, or NO₃, and “a” stands for a number from 0.3 to 5, preferably from 0.8 to 2.5, in particular from 1 to 2, so that the molar ratio of Al to X is 0.9:1 to 2.1:1. These types of activated antiperspirant aluminum salts are disclosed in the publication U.S. Pat. No. 6,074,632 A, for example. Aluminum chlorohydrate (i.e., X stands for Cl in the above formula) is particularly preferred, and in particular, 5/6-basic aluminum chlorohydrate where “a”=1, so that the molar ratio of aluminum to chlorine is 1.9:1 to 2.1:1.

Preferred activated aluminum-zirconium salts are those of general formula ZrO(OH)_{2-pb}Y_{b}, where Y stands for Cl, Br, I, NO₃, or SO₄, b stands for a rational number from 0.8 to 2, and p stands for the valence of Y, so that the Al:Zr molar ratio is 2 to 10, and the metal: (X+Y) ratio is 0.73 to 2.1, preferably 0.9 to 1.5. These types of activated antiperspirant aluminum-zirconium salts are disclosed in the above-mentioned publication U.S. Pat. No. 6,074,632 A, for example. One particularly preferred salt is aluminum-zirconium chlorohydrate (i.e., X and Y stand for Cl), which has an Al:Zr ratio of 2 to 10 and a molar metal: Cl ratio of 0.9 to 2.1. Preferred antiperspirant active substances are disclosed in the publications U.S. Pat. No. 6,663,854 A and US 2004/0009133 A1.

Antiperspirant aluminum salts particularly preferred according to the invention have a molar metal-to-chloride ratio of 1.9 to 2.1. The metal-to-chloride ratio of aluminum sesquichlorohydrates, likewise particularly preferred within the scope of the invention, is 1.5:1 to 1.8:1. Preferred aluminum-zirconium tetrachlorohydrates have an Al:Zr molar ratio of 2 to 6 and a metal:chloride molar ratio of 0.9 to 1.3, in particular salts having a molar metal-to-chloride ratio of 0.9 to 1.1, preferably 0.9 to 1.0, being preferred.

Within the scope of the present invention, particularly high activity and significantly improved stabilization of the antiperspirant aluminum salt is achieved when the compound of formula (M-I) is contained in a total quantity of 0.05 to 8% by weight, preferably 0.1 to 7% by weight, more preferably 0.3 to 5% by weight, even more preferably 0.5 to 3% by weight, very particularly preferably 0.8 to 2.5% by weight, in particular 1 to 2% by weight, based on the total weight of the cosmetic antiperspirant agent. In addition, within the scope of the present invention it may be provided that the compound of formula (M-I) is contained in a total quantity of 0.05 to 35% by weight, preferably 0.5 to 33% by weight, more preferably 1 to 31% by weight, even more preferably 3 to 29% by weight, particularly preferably 5 to 27% by weight, in particular 8 to 25% by weight, based on the total weight of the cosmetic antiperspirant agent. However, within the scope of the invention it is also possible for the cosmetic antiperspirant agents to contain the compound of formula (M-I) in a total quantity of 0.05 to 20% by weight, preferably 0.3 to 18% by weight, more preferably 0.5 to 15% by weight, even more preferably 0.7 to 10% by weight, particularly preferably 1.0 to 9% by weight, in particular 1.5 to 6% by weight, based on the total weight of the propellant-containing cosmetic antiperspirant agent. Without being restricted to this theory, use of the above-mentioned quantities of the compound of formula (M-I) results in significantly increased formation of short-chain polymers of the antiperspirant aluminum salt, and thus, in an improved antiperspirant effect. Furthermore, the short-chain polymers of the antiperspirant aluminum salt itself are effectively stabilized over a long time period in the presence of protic solvents, so that use of the compound of formula (M-I) in the quantities stated above results in a significantly improved antiperspirant effect, even after an extended storage period.

Particularly good results with regard to improvement of the antiperspirant effect are obtained when the cosmetic antiperspirant agent contains at least one compound of formula (M-Ia) and/or (M-Ib)

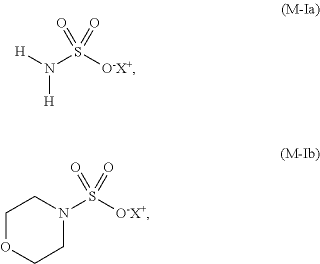

where X⁺ stands for H⁺, Li⁺, Na⁺, K⁺, NH₄⁺, ½Mg²⁺, ½Ca²⁺, ½Mn²⁺, ½Zn²⁺, ⅓Al³⁺, ¼Zr⁴⁺, or at least one antiperspirant aluminum salt. The combination or mixture of amidosulfonic acid or the salts thereof (formula M-Ia) and/or of morpholinosulfonic acid or the salts thereof (formula M-Ib) with an antiperspirant aluminum salt results in increased activation of the antiperspirant aluminum salts, and also results in particularly effective stabilization of the short-chain polymers that are formed, so that the cosmetic antiperspirant agents according to the invention have a significantly improved antiperspirant effect.

The antiperspirant effect of the cosmetic antiperspirant agents according to the invention may be further improved when the cosmetic antiperspirant agent has a weight ratio of the antiperspirant aluminum salt to the compound of formula (M-I) of 40:1 to 19:1, preferably 30:1 to 16:1, more preferably 20:1 to 15:1, even more preferably 10:1 to 13:1, particularly preferably 9:1 to 12:1, in particular 8:1 to 1:1. The above-mentioned weight ratio refers to the total quantity of all antiperspirant aluminum salts and the total weight of all compounds of formula (M-I) in the cosmetic antiperspirant agent.

Within the scope of one particularly preferred embodiment, the weight ratio of the antiperspirant aluminum salt to the compound of formula (M-I) is 5:1. In addition, use of a weight ratio of the antiperspirant aluminum salt to the compound of formula (M-I) of 10:1 also results in cosmetic antiperspirant agents according to the invention which have an excellent antiperspirant effect and for which no significant deterioration of the antiperspirant effect occurs, even after long storage periods. In addition, when the above-mentioned weight ratio is used, protic solvents may also be used, in particular in high concentrations or quantities, without adversely affecting the antiperspirant effect of the cosmetic antiperspirant agents according to the invention, so that extremely flexible packaging of the agents according to the invention is possible.

According to another embodiment of the present invention, the cosmetic antiperspirant agent contains no zirconium-containing compounds. Avoiding the use according to the invention of zirconium-containing antiperspirant compounds such as zirconium-aluminum mixed salts results in more cost-effective preparation of the cosmetic antiperspirant agents according to the invention, since the raw materials for producing the zirconium-containing compounds are more expensive.

The cosmetic antiperspirant agent preferably contains free water in a total quantity of less than 10% by weight, preferably less than 8% by weight, more preferably less than 5% by weight, even more preferably less than 3% by weight, particularly preferably less than 1% by weight, in particular 0% by weight, based on the total weight of the cosmetic antiperspirant agent. Within the meaning of the present invention, free water is thus understood to mean water that is different from water of crystallization, water of hydration, or similarly molecularly bound water of the components used, in particular the antiperspirant aluminum salts.

It has surprisingly been found that, for a combination of an antiperspirant aluminum salt with a compound of formula (M-I), the quantity of short-chain polymers may be significantly increased when the cosmetic antiperspirant agents according to the invention contain free water in a quantity of 15 to 96% by weight, based on the total weight of the cosmetic antiperspirant agent. In one particularly preferred embodiment of the present invention, the cosmetic antiperspirant agent therefore contains free water in a total quantity of 15 to 96% by weight, preferably 25 to 80% by weight, more preferably 30 to 70% by weight, in particular 40 to 60% by weight, based on the total weight of the cosmetic antiperspirant agent.

The cosmetic antiperspirant agents according to the invention are preferably present as a suspension of the undissolved antiperspirant aluminum salt in the cosmetic oil which is liquid at 20° C. and 1013 hPa.

In another preferred administration form, the cosmetic antiperspirant agent is present as a water-in-oil emulsion. This may in particular be a sprayable water-in-oil emulsion which may be sprayed by means of a propellant. In this regard, it is preferred when the cosmetic antiperspirant agent according to the invention, which is present in the form of a water-in-oil emulsion, contains the compound of formula (M-I) in a total quantity of 0.05 to 8% by weight, preferably 0.1 to 7% by weight, more preferably 0.3 to 5% by weight, even more preferably 0.5 to 3% by weight, particularly preferably 0.8 to 2.5% by weight, in particular 1 to 2% by weight, based on the total weight of the cosmetic antiperspirant agent.

Within the scope of the present invention, it may also be provided for the cosmetic antiperspirant agent to be present as an oil-in-water emulsion. In this case, the agent according to the invention is preferably sprayed as a propellant-free pump spray or squeeze spray or applied as a roll-on. In this regard, it is preferred when the cosmetic antiperspirant agent, present in the form of an oil-in-water emulsion, contains the compound of formula (M-I) in a total quantity of 0.05 to 8% by weight, preferably 0.1 to 7% by weight, more preferably 0.3 to 5% by weight, even more preferably 0.5 to 3% by weight, particularly preferably 0.8 to 2.5% by weight, in particular 1 to 2% by weight, based on the total weight of the cosmetic antiperspirant agent.

According to the invention, it may also be provided for the cosmetic antiperspirant agent to be present as an aqueous, aqueous-alcoholic, or aqueous-glycolic solution. Due to the combination according to the invention of an antiperspirant aluminum salt with a compound of formula (M-I), large quantities of activated aluminum salt may be formed or stabilized, even in protic solvents such as aqueous solutions, so that according to the invention, the use of aqueous cosmetic antiperspirant agents is also possible without deactivation of the antiperspirant aluminum salts, and thus, significant reduction of the antiperspirant effect, occurring.

According to one preferred embodiment of the present invention, the cosmetic antiperspirant agent contains ethanol in a total quantity of 1 to 50% by weight, preferably 5 to 40% by weight, more preferably 7 to 35% by weight, in particular 10 to 30% by weight, based on the total weight of the cosmetic antiperspirant agent. However, it may also be provided that the cosmetic antiperspirant agent contains ethanol in a total quantity of 10 to 95% by weight, preferably 15 to 90% by weight, more preferably 20 to 87% by weight, even more preferably 30 to 85% by weight, in particular 40 to 80% by weight, based on the total weight of the cosmetic antiperspirant agent. As stated above, by use of the compound of formula (M-I), even large quantities of protic solvents such as ethanol may be used without adversely affecting the activation or the stabilization of the short-chain polymers of the antiperspirant aluminum salt.

The cosmetic antiperspirant agent according to the invention may be applied using various methods. According to one preferred embodiment, the cosmetic antiperspirant agent is provided as a spray application. The spray application takes place using a spray device, which in a container has a filling of the liquid, viscous-flowable, cosmetic antiperspirant agent according to the invention in the form of a suspension or powder. The filling may be under the pressure of a propellant (compressed gas cans, compressed gas packages, aerosol packages), or may be a mechanically operated pump atomizer without propellant (pump sprays/squeeze bottles). The containers have a dispensing device, preferably in the form of valves, which allow the contents to be dispensed as a mist, fog, foam, powder, paste, or liquid jet. Cylindrical vessels made of metal (aluminum, tinplate, maximum capacity preferably 1000 mL), safety glass or shatterproof glass or plastic (maximum capacity preferably 220 mL), or nonshatterproof glass or plastic (maximum capacity preferably 50 to 400 mL) are primarily suitable as containers for the spray devices. Agents in the form of a cream, gel, paste, or liquid may be packaged, for example, in pump, spray, or squeeze dispensers, in particular also in multichamber pump, multichamber spray, or multichamber squeeze dispensers. The packaging for the agents according to the invention may be opaque, transparent, or translucent.

The cosmetic antiperspirant agent is preferably provided as a stick, soft solid, cream, roll-on, dibenzylidene alditol-based gel, or a loose or compact powder. The formulation of the cosmetic antiperspirant agent according to the invention in a certain administration form, for example an antiperspirant roll-on, an antiperspirant stick, or an antiperspirant gel, preferably depends on the requirements of the intended use. Depending on the intended use, the cosmetic antiperspirant agents according to the invention may therefore be present in solid, semisolid, liquid, dispersed, emulsified, suspended, gel, multiphase, or powdered form. Within the meaning of the present invention, the term “liquid” also includes any types of dispersions of solids in liquids. Furthermore, within the meaning of the present invention, mutliphase cosmetic antiperspirant agents according to the invention are understood to mean agents having at least two different phases with a phase separation, and in which the phases may be situated horizontally, i.e., one above the other, or vertically, i.e., next to one another.

The application may take place with a roller ball applicator, for example. Such rollers have a ball which is supported in a ball bed and which may be moved over a surface. In the process, the ball takes up a portion of the antiperspirant agent to be distributed and delivers it to the surface to be treated. As stated above, the packaging for the agents according to the invention may be opaque, transparent, or translucent.

Furthermore, it is also possible to apply the cosmetic antiperspirant agents according to the invention by means of a solid stick.

However, it may also be preferred according to the invention for the cosmetic antiperspirant agent to be contained on and/or in a disposable substrate selected from the group of wipes, pads, and daubs. Particularly preferred are wet wipes, i.e., wet wipes which are prepackaged, preferably individually packaged, for the user, which are well known, for example, from the area of glass cleaning or from the area of wet toilet wipes. Such wet wipes, which advantageously may also contain preservatives, are impregnated or loaded with a cosmetic antiperspirant agent according to the invention and preferably individually packaged. They may be used as a deodorant wipe, for example, a use which is presently of particular interest. Preferred substrate materials are selected from porous flat wipes. They may be made of a fibrous or cellular flexible material which has sufficient mechanical stability, and at the same time, softness for use on the skin. These wipes include wipes made of woven and nonwoven (fleece) synthetic and natural fibers, felt, paper, or foam, such as hydrophilic polyurethane foam. According to the invention, preferred deodorizing or antiperspirant substrates may be obtained by soaking or impregnating, or also fusing, a cosmetic antiperspirant agent according to the invention on a substrate.

The cosmetic antiperspirant agents according to the invention may also contain further auxiliary substances. The cosmetic antiperspirant agents according to the invention preferably contain at least one further auxiliary substance selected from the group of (i) emulsifiers and/or surfactants; (ii) hydrogel-forming agents; (iii) chelate-forming agents; (iv) deodorant active substances; (v) monohydric and/or polyhydric alcohols and/or polyethylene glycols; (vi) skin-cooling active substances; (vii) propellants; (viii) thickeners, and (ix) the mixtures thereof.

Emulsifiers and surfactants which are preferably suitable according to the invention are selected from anionic, cationic, nonionic, amphoteric, in particular ampholytic and zwitterionic, emulsifiers and surfactants. Surfactants are amphiphilic (bifunctional) compounds composed of at least one hydrophobic and at least one hydrophilic molecular portion. The hydrophobic moiety is preferably a hydrocarbon chain having 8 to 28 carbon atoms, which may be saturated or unsaturated, linear or branched. This C₈-C₂₈ alkyl chain is particularly preferably linear.

Anionic surfactants are understood to mean surfactants having only anionic charges; they contain carboxyl groups, sulfonic acid groups, or sulfate groups, for example. Particularly preferred anionic surfactants are alkyl sulfates, alkyl ether sulfates, acylglutamates, and C₈₋₂₄ carboxylic acids and the salts thereof, the so-called soaps.

Cationic surfactants are understood to mean surfactants having only cationic charges; they contain quaternary ammonium groups, for example. Cationic surfactants of the quaternary ammonium compound, esterquat, and amidoamine types are preferred. Preferred quaternary ammonium compounds are ammonium halides as well as the imidazolium compounds known under the INCI names Quaternium-27 and Quaternium-83. Quaternized protein hydrolysates are further cationic surfactants which are usable according to the invention. Preferred esterquats are quaternized ester salts of fatty acids with triethanolamine, quaternized ester salts of fatty acids with diethanolalkylamines, and quaternized ester salts of fatty acids with 1,2-dihydroxypropyldialkylamines.

The amphoteric surfactants are divided into ampholytic surfactants and zwitterionic surfactants. Ampholytic surfactants are understood to mean those surface-active compounds which have acidic (—COOH or —SO₃H groups, for example) as well as basic (amino groups, for example) hydrophilic groups and which thus have acidic or basic behavior, depending on the conditions. Zwitterionic surfactants are understood by those skilled in the art to be surfactants bearing a negative charge and a positive charge in the same molecule. Examples of preferred zwitterionic surfactants are betaines, N-alkyl-N,N-dimethylammonium glycinates, N-acyl-aminopropyl-N,N-dimethylammonium glycinates, and 2-alkyl-3-carboxymethyl-3-hydroxyethyl imidazolines, in each case having 8 to 24 carbon atoms in the alkyl group. Examples of preferred ampholytic surfactants are N-alkylglycines, N-alkylaminopropionic acids, N-alkylaminobutyric acids, N-alkyliminodipropionic acids, N-hydroxyethyl-N-alkylamidopropylglycine, N-alkyltaurines, N-alkylsarcosines, 2-alkylaminopropionic acids, and alkylaminoacetic acids, in each case having 8 to 24 carbon atoms in the alkyl group.

The compositions according to the invention, which are formulated as an emulsion, in particular as an oil-in-water emulsion, preferably contain at least one nonionic oil-in-water emulsifier having an HLB value of greater than 7 to 20. These are emulsifiers generally known to those skilled in the art, which are listed, for example, in Kirk-Othmer, Encyclopedia of Chemical Technology, 3rd Edition, 1979, Volume 8, pages 913-916. For ethoxylated products, the HLB value is calculated according to the formula HLB=(100−L):5, where L is the weight fraction of the lipophilic groups, i.e., the fatty alkyl or fatty acyl groups, in the ethylene oxide adducts, expressed in percent by weight. In this regard, it may be preferred according to the invention when a water-in-oil emulsifier having an HLB value of greater than 1.0 and less than or equal to 7.0 is also used. Within the scope of the present invention, suitable nonionic oil-in-water emulsifiers and suitable nonionic water-in-oil emulsifiers are described in German Unexamined Patent Application DE 102006004957 A1, for example.

For thickening the cosmetic antiperspirant agents according to the invention, hydrogel-forming substances are preferably used which are selected from cellulose ethers, primarily hydroxyalkylcelluloses, in particular hydroxypropylcellulose, hydroxypropylmethylcellulose, hydroxyethylcellulose, carboxymethylcellulose, cetylhydroxyethylcellulose, hydroxybutylmethylcellulose, methylhydroxyethylcellulose, as well as xanthan gum, sclerotium gum, succinoglycans, polygalactomannans, in particular guar gums and locust bean gum, in particular guar gum and locust bean gum themselves, and the nonionic hydroxyalkyl guar derivatives and locust bean gum derivatives, such as hydroxypropyl guar, carboxymethylhydroxypropyl guar, hydroxypropylmethyl guar, hydroxyethyl guar, and carboxymethyl guar, as well as pectins, agar, carrageenan, tragacanth, gum arabic, karaya gum, tara gum, gellan, gelatins, casein, propylene glycol alginate, alginic acids and the salts thereof, in particular sodium alginate, potassium alginate, and calcium alginate, as well as polyvinylpyrrolidones, polyvinyl alcohols, polyacrylamides, and, although less preferred, physically modified starches (by pregelatinization, for example) and/or chemically modified starches, in particular hydroxypropylated starch phosphates and octenyl starch succinates and the aluminum, calcium, or sodium salts thereof, and also, although less preferred, acrylic acid-acrylate copolymers, acrylic acid-acrylamide copolymers, acrylic acid-vinylpyrrolidone copolymers, acrylic acid-vinyl formamide copolymers, and polyacrylates. Particularly preferred hydrogel-forming agents are selected from cellulose ethers, primarily hydroxyalkylcelluloses, in particular hydroxypropylcellulose, hydroxypropylmethylcellulose, hydroxyethylcellulose, carboxymethylcellulose, cetylhydroxyethylcellulose, hydroxybutylmethylcellulose, and methylhydroxyethylcellulose, and mixtures thereof. Hydroxyethylcellulose is preferably used as the hydrogel-forming agent.

To further assist in the activating effect of the compound of formula (M-I), it may be advantageous to add to the cosmetic antiperspirant agents according to the invention at least one chelate-forming agent that is selected from ethylenediaminetetraacetic acid (EDTA) and the salts thereof, and from nitrilotriacetic acid (NTA) and mixtures of these substances, in a total quantity of 0.01 to 0.5% by weight, preferably 0.02 to 0.3% by weight, in particular 0.05 to 0.1% by weight, based on the total weight of the antiperspirant agent according to the invention. Within the scope of the present invention, however, chelate-forming agents may also be used which are selected from the group of β-alanindiacetic acid, cyclodextrin, diethylenetriaminepentamethylene phosphonic acid, sodium, potassium, and calcium disodium, ammonium, and triethanolamine salts of ethylenediaminetetraacetic acid (EDTA), etidronic acid, hydroxyethylethylenediaminetetraacetic acid (HEDTA) and the sodium salts thereof, sodium salts of nitrilotriacetic acid (NTA), diethylenetriaminepentaacetic acid, and phytic acid, hydroxypropylcyclodextrin, methylcyclodextrin, aminotrimethylene phosphonate pentasodium, ethylenediaminetetramethylene phosphonate pentasodium, diethylenetriaminepentaacetate pentasodium, pentasodium triphosphate, potassium EDTMP, sodium EDTMP, sodium dihydroxyethyl glycinate, sodium phytate, sodium polydimethylglycinophenolsulfonate, tetrahydroxyethylethylenediamine, tetrahydroxypropylethylenediamine, tetrapotassium etidronate, tetrasodium etidronate, tetrasodiumimino disuccinate, trisodiumethylenediamine disuccinate, and desferrioxamine.

The antiperspirant effect of the cosmetic antiperspirant agents according to the invention may be further increased when at least one deodorant active substance is contained in a total quantity of 0.0001 to 40% by weight, preferably 0.2 to 20% by weight, more preferably 1 to 15% by weight, in particular 1.5 to 5% by weight, based on the total weight of the cosmetic antiperspirant agent according to the invention. If ethanol is used in the agents according to the invention, within the scope of the present invention it is considered not as a deodorant active substance, but, rather, as a component of the carrier. Deodorant active substances preferred according to the invention are selected from the group of (i) silver salts; (ii) aromatic alcohols, in particular 2-benzylheptan-1-ol and mixtures of 2-benzylheptan-1-ol and phenoxyethanol; (iii) 1,2-alkanediols having 5 to 12 carbon atoms, in particular 3-(2-ethylhexyloxy)-1,2-propanediol; (iv) triethyl citrates; (v) active substances against exoesterases, in particular against aryl sulfatases, lipases, beta-glucuronidases, and cystathionine β-lyases; (vi) cationic phospholipids; (vii) odor absorbers, in particular silicates such as montmorillonite, kaolinite, illite, beidellite, nontronite, saponite, hectorite, bentonite, smectite, and talc, zeolites, zinc ricinoleate, cyclodextrins; (viii) ion exchangers having a deodorizing action; (ix) antibacterial agents; (x) prebiotically active components; and (xi) mixtures of these deodorant active substances.

Preferred cosmetic antiperspirant agents according to the invention also contain at least one water-soluble polyhydric C₂₋₉ alkanol having 2 to 6 hydroxyl groups and/or at least one water-soluble polyethylene glycol having 3 to 50 ethylene oxide units, and mixtures thereof. These do not include the above-mentioned deodorant active substances in the form of 1,2-alkanediols. Preferred alkanols are selected from 1,2-propylene glycol, 2-methyl-1,3-propanediol, glycerin, 1,2-butylene glycol, 1,3-butylene glycol, 1,4-butylene glycol, pentylene glycols such as 1,2-pentanediol and 1,5-pentanediol, hexanediols such as 1,2-hexanediol and 1,6-hexanediol, hexanetriols such as 1,2,6-hexanetriol, 1,2-octanediol, 1,8-octanediol, dipropylene glycol, tripropylene glycol, diglycerin, triglycerin, erythrite, sorbitol, cis-1,4-dimethylolcyclohexane, trans-1,4-dimethylolcyclohexane, any given isomeric mixtures of cis- and trans-1,4-dimethylolcyclohexane, and mixtures of the above-mentioned substances. Suitable water-soluble polyethylene glycols are selected from PEG-3, PEG-4, PEG-6, PEG-7, PEG-8, PEG-9, PEG-10, PEG-12, PEG-14, PEG-16, PEG-18, and PEG-20, and mixtures thereof, with PEG-3 to PEG-8 being preferred.

According to another embodiment of the present invention, the cosmetic antiperspirant agents also contain at least one skin-cooling active substance. Examples of skin-cooling active substances which are suitable according to the invention are menthol, isopulegol, and menthol derivatives, for example menthyl lactate, menthyl glycolate, menthyl ethyl oxamate, menthylpyrrolidone carboxylic acid, menthyl methyl ether, menthoxypropanediol, menthone glycerin acetal (9-methyl-6-(1-methylethyl)-1,4-dioxaspiro(4.5)decane-2-methanol), monomenthyl succinate, 2-hydroxymethyl-3,5,5-trimethylcyclohexanol, and 5-methyl-2-(1-methylethyl)cyclohexyl-N-ethyloxamate. Preferred as skin-cooling active substances are menthol, isopulegol, menthyl lactate, menthoxypropanediol, menthylpyrrolidone carboxylic acid, and 5-methyl-2-(1-methylethyl)cyclohexyl-N-ethyloxamate and mixtures of these substances, in particular mixtures of menthol and menthyl lactate, of menthol, menthol glycolate, and menthyl lactate, of menthol and menthoxypropanediol, or of menthol and isopulegol.

In addition, it may be provided that the cosmetic antiperspirant agents according to the invention contain a propellant. In this case, these agents are provided as a propellant gas-driven aerosol. Preferred propellants (propellant gases) are propane, propene, n-butane, isobutane, isobutene, n-pentane, pentene, isopentane, isopentene, methane, ethane, dimethyl ether, nitrogen, air, oxygen, nitrous oxide, 1,1,1,3-tetrafluoroethane, heptafluoro-n-propane, perfluoroethane, monochlordifluoromethane, 1,1-difluoroethane, tetrafluoropropene, individually as well as in the mixtures thereof. Hydrophilic propellant gases, for example carbon dioxide, may also be advantageously used within the meaning of the present invention when the proportion of hydrophilic gases is selected to be small, and lipophilic propellant gas (propane/butane, for example) is present in excess. Propane, n-butane, isobutane, and mixtures of these propellant gases are particularly preferred. It has been shown that the use of n-butane as the sole propellant gas may be particularly preferred according to the invention. The total quantity of propellant is 20 to 95% by weight, preferably 30 to 85% by weight, in particular 40 to 75% by weight, in each case based on the total weight of the antiperspirant composed of the cosmetic antiperspirant agent according to the invention and the propellant. In this regard, it may also be provided that the total quantity of propellant is 1 to 95% by weight, preferably 2 to 85% by weight, in particular 3 to 75% by weight, in each case based on the total weight of the antiperspirant composed of the cosmetic antiperspirant agent according to the invention and the propellant.

According to the invention, lipophilic thickeners may also be used as auxiliary substances. The at least one antiperspirant aluminum salt is preferably suspended undissolved in at least one cosmetic oil which is liquid at 20° C. and 1013 hPa. For better application, at least one lipophilic thickener may also be added to the suspension as a suspension aid. Lipophilic thickeners preferred according to the invention are selected from hydrophobized clay materials and pyrogenic silicic acids.

In one preferred embodiment, the cosmetic antiperspirant agents according to the invention are characterized in that they are provided as a water-in-oil emulsion and contain, based on the total weight of the cosmetic antiperspirant agent according to the invention,
at least one antiperspirant aluminum salt in a total quantity of 2 to 75% by weight, preferably 3 to 70% by weight, more preferably 4 to 65% by weight, particularly preferably 5 to 55% by weight, even more preferably 8 to 40% by weight, in particular 10 to 30% by weight,
at least one compound of formula (M-I) in a total quantity of 0.05 to 8% by weight, preferably 0.1 to 7% by weight, more preferably 0.3 to 5% by weight, particularly preferably 0.5 to 3% by weight, even more preferably 0.8 to 2.5% by weight, in particular 1 to 2% by weight,
12 to 90% by weight, preferably 25 to 55% by weight, more preferably 30 to 50% by weight, in particular 35 to 45% by weight, of water,
at least one emulsifier, and
at least one substance selected from the group of cosmetic oils which are liquid at 20° C. and 1013 hPa, odorants, and waxes.

In another preferred embodiment, the cosmetic antiperspirant agents according to the invention are characterized in that they are provided as an oil-in-water emulsion and contain, based on the total weight of the cosmetic antiperspirant agent according to the invention,
at least one antiperspirant aluminum salt in a total quantity of 2 to 75% by weight, preferably 3 to 70% by weight, more preferably 4 to 65% by weight, particularly preferably 5 to 55% by weight, even more preferably 8 to 40% by weight, in particular 10 to 30% by weight,
at least one compound of formula (M-I) in a total quantity of 0.05 to 8% by weight, preferably 0.1 to 7% by weight, more preferably 0.3 to 5% by weight, particularly preferably 0.5 to 3% by weight, even more preferably 0.8 to 2.5% by weight, in particular 1 to 2% by weight,
15 to 90% by weight, preferably 25 to 55% by weight, more preferably 30 to 50% by weight, in particular 35 to 45% by weight, of water,
at least one emulsifier, and
at least one substance selected from the group of cosmetic oils which are liquid at 20° C. and 1013 hPa, odorants, and waxes.

Another preferred embodiment of the present invention includes cosmetic antiperspirant agents according to the invention which are characterized in that they contain, based on the total weight of the cosmetic antiperspirant agent according to the invention,
at least one antiperspirant aluminum salt in a total quantity of 2 to 75% by weight, preferably 3 to 70% by weight, more preferably 4 to 65% by weight, particularly preferably 5 to 55% by weight, even more preferably 8 to 40% by weight, in particular 10 to 30% by weight,
at least one compound of formula (M-I) in a total quantity of 0.05 to 8% by weight, preferably 0.1 to 7% by weight, more preferably 0.3 to 5% by weight, particularly preferably 0.5 to 3% by weight, even more preferably 0.8 to 2.5% by weight, in particular 1 to 2% by weight,
15 to 90% by weight, preferably 25 to 80% by weight, more preferably 30 to 75% by weight, in particular 40 to 60% by weight, of water, and
0.01 to 2% by weight, preferably 0.1 to 1% by weight, more preferably 0.2 to 0.7% by weight, in particular 0.3 to 0.5% by weight, of a hydrogel-forming substance.

Another preferred embodiment of the present invention includes cosmetic antiperspirant agents according to the invention which are characterized in that they contain, based on the total weight of the cosmetic antiperspirant agent according to the invention,
at least one antiperspirant aluminum salt in a total quantity of 2 to 75% by weight, preferably 3 to 70% by weight, more preferably 4 to 65% by weight, particularly preferably 5 to 55% by weight, even more preferably 8 to 40% by weight, in particular 10 to 30% by weight,
at least one compound of formula (M-I) in a total quantity of 0.05 to 8% by weight, preferably 0.1 to 7% by weight, more preferably 0.3 to 5% by weight, particularly preferably 0.5 to 3% by weight, even more preferably 0.8 to 2.5% by weight, in particular 1 to 2% by weight,
15 to 90% by weight, preferably 25 to 80% by weight, more preferably 30 to 75% by weight, in particular 40 to 60% by weight, of water, and
0.01 to 2% by weight, preferably 0.1 to 1% by weight, more preferably 0.2 to 0.7% by weight, in particular 0.3 to 0.5% by weight, of a hydrogel-forming substance,
   wherein the cosmetic antiperspirant agents according to the invention have a dynamic viscosity in the range of 10 to 5000 mPas, preferably 100 to 1000 mPas, more preferably 200 to 800 mPas, measured with a Brookfield viscosimeter, spindle RV 4, 20 s⁻¹, no helipath, at 20° C. ambient temperature and 20° C. sample temperature.

Another preferred embodiment of the present invention includes cosmetic antiperspirant agents according to the invention which are characterized in that they contain, based on the total weight of the cosmetic antiperspirant agent according to the invention,
at least one antiperspirant aluminum salt in a total quantity of 2 to 75% by weight, preferably 3 to 70% by weight, more preferably 4 to 65% by weight, particularly preferably 5 to 55% by weight, even more preferably 8 to 40% by weight, in particular 10 to 30% by weight,
at least one compound of formula (M-I) in a total quantity of 0.05 to 8% by weight, preferably 0.1 to 7% by weight, more preferably 0.3 to 5% by weight, particularly preferably 0.5 to 3% by weight, even more preferably 0.8 to 2.5% by weight, in particular 1 to 2% by weight,
15 to 90% by weight, preferably 25 to 80% by weight, more preferably 30 to 75% by weight, in particular 40 to 60% by weight, of water, and
0.01 to 2% by weight, preferably 0.1 to 1% by weight, more preferably 0.2 to 0.7% by weight, in particular 0.3 to 0.5% by weight, of a hydrogel-forming substance,
   wherein the cosmetic antiperspirant agents according to the invention have a dynamic viscosity in the range of 1000 to 800,000 mPas, preferably 2000 to 700,000 mPas, more preferably 3000 to 500,000 mPas, measured with a Brookfield viscosimeter, spindle RV 4, 20 s⁻¹, no helipath, at 20° C. ambient temperature and 20° C. sample temperature.

In another preferred embodiment, the cosmetic antiperspirant agents according to the invention are characterized in that they are provided as a water-in-oil emulsion and contain, based on the total weight of the cosmetic antiperspirant agent according to the invention,
at least one antiperspirant aluminum salt in a total quantity of 2 to 75% by weight, preferably 3 to 70% by weight, more preferably 4 to 65% by weight, particularly preferably 5 to 55% by weight, even more preferably 8 to 40% by weight, in particular 10 to 30% by weight,
at least one compound of formula (M-I) in a total quantity of 0.05 to 8% by weight, preferably 0.1 to 7% by weight, more preferably 0.3 to 5% by weight, particularly preferably 0.5 to 3% by weight, even more preferably 0.8 to 2.5% by weight, in particular 1 to 2% by weight,
15 to 75% by weight, preferably 25 to 60% by weight, more preferably 30 to 55% by weight, in particular 35 to 50% by weight, of water.

In another preferred embodiment, the cosmetic antiperspirant agents according to the invention are characterized in that they are provided as an oil-in-water emulsion and contain, based on the total weight of the cosmetic antiperspirant agent according to the invention,
at least one antiperspirant aluminum salt in a total quantity of 2 to 75% by weight, preferably 3 to 70% by weight, more preferably 4 to 65% by weight, particularly preferably 5 to 55% by weight, even more preferably 8 to 40% by weight, in particular 10 to 30% by weight,
at least one compound of formula (M-I) in a total quantity of 0.05 to 8% by weight, preferably 0.1 to 7% by weight, more preferably 0.3 to 5% by weight, particularly preferably 0.5 to 3% by weight, even more preferably 0.8 to 2.5% by weight, in particular 1 to 2% by weight,
15 to 90% by weight, preferably 25 to 80% by weight, more preferably 30 to 75% by weight, in particular 40 to 60% by weight, of water, and
optionally 0.01 to 2% by weight, preferably 0.1 to 1% by weight, more preferably 0.2 to 0.7% by weight, in particular 0.3 to 0.5% by weight, of a hydrogel-forming substance.

A second subject matter of the present invention is the use of a combination of at least one substance selected from the group of cosmetic oils which are liquid at 20° C. and 1013 hPa, odorants, and waxes, at least one antiperspirant aluminum salt in a total quantity of 1 to 80% by weight, based on the total weight of the combination, and at least one compound of formula (M-I)

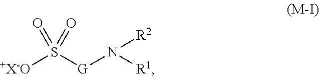

where
G stands for a direct bond or a linear or branched alkylene group having 1 to 6 carbon atoms,
R¹ and R² independently stand for hydrogen or a linear or branched, saturated or unsaturated alkylene group having 1 to 10 carbon atoms, or R¹ and R² form a cyclic group having 5 to 7 carbon atoms and at least one heteroatom selected from the group of N, O, and S, and
X⁺ stands for H⁺, Li⁺, Na⁺, K⁺, NH₄⁺, ½Mg²⁺, ½Ca²⁻, ½Mn²⁻, ½Zn²⁺, ⅓Al³⁺, ¼Zr⁴⁺, or at least one antiperspirant aluminum salt,
   for reducing and/or preventing perspiration. Within the meaning of the present invention, the term “combination” likewise includes a mixture of the at least one substance and the antiperspirant aluminum salt with a compound of formula (M-I). The statements made with regard to the cosmetic antiperspirant agents according to the invention apply mutatis mutandis to the use of the above-mentioned combination.

Moreover, a third subject matter of the present invention is a nontherapeutic cosmetic method for preventing and/or reducing perspiration of the body, in which a cosmetic antiperspirant agent including at least one substance selected from the group of cosmetic oils which are liquid at 20° C. and 1013 hPa, odorants, and waxes, at least one antiperspirant aluminum salt in a total quantity of 1 to 80% by weight, based on the total weight of the cosmetic antiperspirant agent, and at least one compound of formula (M-I) is applied to the skin, in particular to the skin of the armpits. The statements made with regard to the cosmetic antiperspirant agents according to the invention and the use according to the invention apply mutatis mutandis to the method according to the invention.

Lastly, a fourth subject matter of the present invention is the use of at least one compound of formula (M-I)

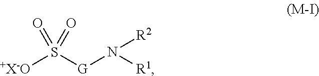

where
G stands for a direct bond or a linear or branched alkylene group having 1 to 6 carbon atoms,
R¹ and R² independently stand for hydrogen or a linear or branched, saturated or unsaturated alkylene group having 1 to 10 carbon atoms, or R¹ and R² form a cyclic group having 5 to 7 carbon atoms and at least one heteroatom selected from the group of N, O, and S, and
X⁺ stands for H⁺, Li⁺, Na⁺, K⁺, NH₄⁺, ½Mg²⁺, ½Ca²⁻, ½Mn²⁻, ½Zn²⁺, ⅓Al³⁺, ¼Zr⁴⁺, or at least one antiperspirant aluminum salt,
   for activating and/or stabilizing at least one antiperspirant aluminum salt.

Within the meaning of the present invention, activation is understood to mean the significantly increased formation of short-chain polymers of the antiperspirant aluminum salt when the compound of formula (M-I) is used.

In addition, within the meaning of the present invention, stabilization is understood to mean the avoidance or significant retardation of the re-formation of higher-chain polymers from the short-chain polymers of the antiperspirant aluminum salts, formed during the activation.

By using particular sulfonic acids of formula (M-I), the activity of antiperspirant aluminum salts has surprisingly been significantly increased, even over an extended storage period. Without being restricted to this theory, a significant increase in the proportion of short-chain species of the antiperspirant aluminum salts is achieved by the use of compounds of formula (M-I). In addition, the short-chain species formed in this way are stabilized very well by the above-mentioned sulfonic acids, thus ensuring an improved antiperspirant effect over a long period of time.

Particularly good activation and/or stabilization of the at least one antiperspirant aluminum salt is achieved when the compound of formula (M-I) is used in a total quantity of 0.01 to 50% by weight, preferably 0.1 to 40% by weight, more preferably 0.5 to 30% by weight, even more preferably 1 to 20% by weight, particularly preferably 1.5 to 10% by weight, in particular 2 to 7% by weight, based on the total weight of the compound of formula (M-I), the at least one antiperspirant aluminum salt, and optionally further ingredients. The quantities mentioned above result in particularly good activation and stabilization of the antiperspirant aluminum salt, even over extended storage periods.

In this regard, it is advantageous when the antiperspirant aluminum salt is used in a total quantity of 1 to 99.99% by weight, preferably 2 to 80% by weight, more preferably 3 to 60% by weight, even more preferably 4 to 50% by weight, in particular 6 to 45% by weight, based on the total weight of the compound of formula (M-I), the at least one antiperspirant aluminum salt, and optionally further ingredients.

It has been shown to be particularly advantageous for the activation and/or stabilization of the at least one antiperspirant aluminum salt when a weight ratio of the antiperspirant aluminum salt to the compound of formula (M-I) is 5000:1 to 1:0.16, preferably 1000:1 to 1:1, more preferably 100:1 to 1:5, even more preferably 50:1 to 1:10, particularly preferably 10:1 to 1:30, in particular 4:1 to 1:50. The weight ratio mentioned above refers to the total quantity of all antiperspirant aluminum salts and of all compounds of formula (M-I) in the cosmetic antiperspirant agent.

Significantly increased activation and/or stabilization of the antiperspirant aluminum salt surprisingly results when the compound of formula (M-I) is used, even in the presence of large quantities of protic solvents, for example water. It is therefore preferred when a total quantity of free water of 1 to 90% by weight, preferably 5 to 80% by weight, more preferably 10 to 70% by weight, even more preferably 12 to 60% by weight, in particular 15 to 55% by weight, based on the total weight of the compound of formula (M-I), the at least one antiperspirant aluminum salt, the free water, and optionally further ingredients, is used.

In addition, it is likewise possible within the scope of the present invention to dry the liquid mixture which results from the activation and stabilization of the at least one antiperspirant aluminum salt, using the compound of formula (M-I). The drying of this mixture may be carried out, for example, by means of conventional drying methods such as spray drying. The powders obtained in this way may be stored very well, and have long storage stability.

The statements made with regard to the cosmetic antiperspirant agents according to the invention apply mutatis mutandis to further preferred embodiments.

The following examples explain the present invention without being limited thereto:

### EXAMPLES

1. HPLC measurements:

For determining the activation of the antiperspirant aluminum salt in the cosmetic antiperspirant agent, the following aqueous solutions were prepared and stored for 4 weeks at room temperature (quantities are expressed in percent by weight):

| | V-I | V-II | E-I** | E-II** | E-III** |
|---|---|---|---|---|---|
| Aluminum chlorohydrate | 10* | 10* | 10* | 10* | 10* |
| (ACH) | | | | | |
| HCl | — | 1 | — | — | — |
| Amidosulfonic acid (M-Ia) | — | — | 0.5 | 1 | 2 |
| Water | 90 | 89 | 89.5 | 89 | 88 |

*active substance
**according to the invention

Such solutions are representative of, among other things, water-containing antiperspirant emulsions (antiperspirant roll-ons; antiperspirant sticks, antiperspirant gels, antiperspirant pump atomizers).

After 4 weeks, the activation resulting directly from the polymer distribution of samples V-I, V-II, E-I, E-II, and E-III was determined by size exclusion chromatography (SEC). For this purpose, the particular sample was initially diluted to the concentration 1 g ACH/25 mL water and filtered (0.2-μm filter). The particular sample was subsequently measured by SEC, using a standard HPLC system with a refractive index detector, using the following parameters:
Flow: 1 mL/min
Temperature: 23° C.
Eluent: 0.02 M HCl
Column: 6.2×250 mm, column material: silanized porous silica microspheres (5 μm).

For each sample, 11, 12, or more peaks were obtained, wherein the peaks with the shorter retention time stand for the long-chain polymers of the ACH, and the peaks with longer retention times stand for polymers of the ACH having shorter chain lengths. The peaks obtained for each sample in the chromatogram were integrated, wherein peaks 2 and 3 (long-chain polymers), 4 to 8 (polymers having an average chain length), 9 to 11 (polymers having a short chain length), and 12 as well as all subsequent peaks (very short chain length of the polymers) were integrated together due to shoulders. The area ratios of the peak groups 1, 2 to 3, 4 to 8, 9 to 11, and 12 and greater are presented in the following table:

| | Peak 1 | Peak 2-3 | Peak 4-8 | Peak 9-11 | Peak 12 and >12 |
|---|---|---|---|---|---|
| V-I | 1.0 | 4.2 | 6.8 | 4.7 | 0 |
| V-II | 1.0 | 2.3 | 3.7 | 2.7 | 0.30 |
| E-I | 1.0 | 3.5 | 5.7 | 5.5 | 0.83 |
| E-II | 1.0 | 3.6 | 7.0 | 8.2 | 0.20 |
| E-III | 1.0 | 4.3 | 12 | 16 | 0.33 |

The formulations E-I, E-II, and E-III according to the invention have a significantly higher proportion of short-chain polymers (peaks 9 to 12 and greater), i.e., much better activation and stabilization, than the comparative examples V-I and V-II. In addition, it is apparent from the above table that the polymer distribution may be shifted toward short-chain polymers by increasing the quantity of amidosulfonic acid. Consequently, an increase in the quantity of amidosulfonic acid results in further improved activation and stabilization of the aluminum salt used.

2. Formulations:

The following formulation examples are intended to explain the subject matter of the invention without being limited thereto. The compound of formula (M-I) used in the following examples is preferably an amidosulfonic acid or the salts thereof (formula (M-Ia)), a morpholinosulfonic acid or the salts thereof (formula (M-Ib)), and the mixtures thereof:

Cosmetic antiperspirant agents according to the invention (quantities are expressed in % by weight)

| | 1 | 2 | 3 | 4 | 5 | 6 |
|---|---|---|---|---|---|---|
| Hydrogenated castor oil | — | — | — | 1.5 | 1.5 | 1.5 |
| Stearyl alcohol | 24.0 | 24.0 | 24.0 | 18 | 18 | 18 |
| Novata AB | — | — | — | 4 | 4 | 4 |
| Powder of ACH and | 15.0 | 22.0 | 20.0 | 11.6 | 15.6 | 12.6 |
| compound of formula | | | | | | |
| (M-I) (spray-dried, | | | | | | |
| contains 10% by | | | | | | |
| weight (M-I)) | | | | | | |
| Al—Zr pentachlorohydrex | 7.00 | — | — | 6.00 | — | — |
| glycine | | | | | | |
| PPG-14 butyl ether | 10.0 | 10.0 | 10.0 | 15.3 | 15.3 | 15.3 |
| Hardened castor oil | 3.0 | 3.0 | 3.0 | — | — | — |
| (e.g., Cutina UR) | | | | | | |
| Myristyl myristate | 1.5 | 1.5 | 1.5 | — | — | — |
| DL-Menthol | 0.2 | 0.2 | 0.2 | — | — | — |
| Eucalyptol | 0.2 | 0.2 | 0.2 | — | — | — |
| Anethol | 0.2 | 0.2 | 0.2 | — | — | — |
| Silica dimethyl silylate | 1.4 | 1.4 | 1.4 | — | — | — |
| Silica | 0.3 | 0.3 | 0.3 | — | — | — |
| Talc | — | — | — | 3 | 3 | 3 |
| Eumulgin ® B1 | — | — | — | 3 | 3 | 3 |
| Fragrance | 2.0 | 2.0 | 2.0 | 1 | 1 | 1 |
| Cyclomethicone (at | ad | ad | ad | ad | ad | ad |
| least 95% by weight | 100 | 100 | 100 | 100 | 100 | 100 |
| cyclopentasiloxane) | | | | | | |

Cosmetic agents according to the invention in the form of an oil-in-water emulsion (quantities are expressed in % by weight)

| | 7 | 8 | 9 |
|---|---|---|---|
| Cutina ® AGS | 2.5 | 2.5 | 2.5 |
| Cutina ® FS45 | 3.5 | 3.5 | 3.5 |
| Eumulgin ® B2 | 0.8 | 0.8 | 0.8 |
| Eumulgin ® B3 | 0.8 | 0.8 | 0.8 |
| Diisopropyl adipate | 6.0 | 6.0 | 6.0 |
| Novata ® AB | 4.0 | 4.0 | 4.0 |
| Cutina ® CP | 5.0 | 5.0 | 5.0 |
| Cutina ® HR | 4.0 | 4.0 | 4.0 |
| Kesterwachs K62 | 5.0 | 5.0 | 5.0 |
| Locron ® L (50% ACH solution) | 40 | 40 | 30 |
| Talc Pharma G | 10 | 10 | 10 |
| Fragrance | 1.2 | 1.2 | 1.2 |
| 2-Benzylheptan-1-ol | — | 0.3 | 0.3 |
| Sensiva SC 50 | 0.6 | 0.6 | 0.6 |
| Compound of formula (M-I) | 2.0 | 3.0 | 3.0 |
| 1,2-Propanediol | 10 | 10 | 10 |
| Water, demineralized | ad 100 | ad 100 | ad 100 |

Cosmetic antiperspirant agents according to the invention in the form of a microemulsion (quantities are expressed in % by weight)

| | 10 | 11 | 12 | 13 |
|---|---|---|---|---|
| Plantaren ® 1200 | 1.7 | 1.7 | — | — |
| Plantaren ® 2000 | 1.1 | 1.4 | 2.4 | 2.4 |
| Glycerin monooleate | 0.71 | 0.71 | — | — |
| Dioctyl ether | 4.0 | 4.0 | 0.090 | 0.090 |
| Octyl dodecanol | 1.0 | 1.0 | 0.020 | 0.020 |
| Fragrance oil | 1.0 | 1.0 | 1.0 | 1.0 |
| Aluminum chlorohydrate | 8.0 | 5.0 | 5.0 | 10 |
| 1,2-Propylene glycol | 5.0 | 5.0 | — | — |
| Glycerin | — | — | 5.0 | 5.0 |
| 2-Benzylheptan-1-ol | 0.50 | — | — | — |
| Triethyl citrate | — | 0.50 | 0.50 | 0.50 |
| Triclosan | 0.10 | — | — | — |
| Compound of formula (M-I) | 1.0 | 2.0 | 2.5 | 2.5 |
| Water | ad 100 | ad 100 | ad 100 | ad 100 |

Cosmetic antiperspirant agents according to the invention in the form of roll-ons (quantities are expressed in % by weight)

| | 14 | 15 | 16 | 17 |
|---|---|---|---|---|
| 96% ethanol (denatured with DEP) | 30 | 30 | 28 | 28 |
| Mergital ® CS 11 | 2.0 | 2.0 | — | — |
| Eumulgin ® B3 | 2.0 | 2.0 | 2.0 | 2.0 |
| Eumulgin ® B1 | — | — | 2.0 | 2.0 |
| 50% aluminum chlorohydrate | — | 20 | — | 16 |
| (Locron L) | | | | |
| Al—Zr pentachlorohydrex glycine | 20 | — | 16 | — |
| Hydroxyethylcellulose | 0.50 | 0.50 | 0.30 | 0.30 |
| Compound of formula (M-I) | 2.5 | 0.50 | 2.0 | 1.5 |
| EDTA | — | — | — | 0.050 |
| Cocamidopropyl PG Dimonium | 0.20 | — | — | — |
| Chloride Phosphate | | | | |
| Fragrance oil | 0.80 | 0.80 | 1.0 | 1.0 |
| Water | ad 100 | ad 100 | ad 100 | ad 100 |

Antiperspirant wipes (Example Nos. 18 to 21)

For the embodiment according to the invention as an antiperspirant wipe, a one-ply substrate made of 100% viscose having a weight per unit area of 50 g/m² was provided in each case with 75 g of the example emulsions 14 or 15 per square meter, or in each case with 75 g of the example compositions 10 or 11, cut into wipes of suitable size, and packed in sachets.

Cosmetic antiperspirant agents according to the invention in the form of a water-in-oil emulsion (quantities are expressed in % by weight)

| | 22 | 23 | 24 |
|---|---|---|---|
| 50% aluminum chlorohydrate in water | 35.6 | 35.6 | 20.0 |
| (Locron L) | | | |
| 1,2-Propylene glycol | 13.0 | 13.0 | 13.0 |
| Cyclohexasiloxane | 6.00 | 6.00 | 6.00 |
| Finsolv TN | 8.00 | 8.00 | 8.00 |
| Abil EM 90 | 1.20 | 1.20 | 1.20 |
| Polyethylene wax | 10.0 | 10.0 | 10.0 |
| (MW = 500 g/mol, Mp = 83-91° C.) | | | |
| Polyalphaolefin wax | 0.100 | 0.100 | 0.100 |
| (MW = 1800 g/mol, Mp = 41° C.) | | | |
| Compound of formula (M-I) | 2.00 | 0.500 | 0.500 |
| EDTA | — | 0.0500 | 0.0500 |
| Water | 25.0 | 25.0 | ad 100 |
| Fragrance | 1.00 | 1.00 | 1.00 |

Cosmetic antiperspirant agents according to the invention (quantities are expressed in % by weight)

| | 25 | 26 |
|---|---|---|
| Cyclopentasiloxane | 14.0 | 14.0 |
| Abil EM 97 | 3.00 | 3.00 |
| 96% ethanol | 10.0 | 10.0 |
| 50% aluminum chlorohydrate in water | 40.0 | 40.0 |
| (Locron L) | | |
| 1,2-Propylene glycol | 20.3 | 20.3 |
| Water | 11.6 | 11.6 |
| Compound of formula (M-I) | 2.00 | 0.500 |
| EDT | — | 0.0750 |
| Fragrance | 1.00 | 1.00 |

Cosmetic antiperspirant agents according to the invention (quantities are expressed in % by weight, based on the total weight of the propellant-free composition)

| | 27 | 28 | 29 | 30 |
|---|---|---|---|---|
| Aluminum chlorohydrate (ACH) | 28.6 | 14.29 | 32.11 | 28.57 |
| Bentone 38 V CG | 5.00 | 3.93 | 4.00 | 5.00 |
| Propylene carbonate | 1.50 | 0.71 | 1.50 | 1.80 |
| Fragrance | 7.14 | 6.50 | 5.00 | 6.50 |
| 2-Ethylhexyl palmitate | — | 73.57 | — | — |
| Abil K 4 | 48.4 | — | — | — |
| Isopropyl myristate | 7.37 | — | 10.00 | 19.22 |
| Triethyl citrate | — | — | 10.5 | 19.2 |
| C₁₀-C₁₃ isoalkanes | — | — | 35.39 | 19.21 |
| Compound of formula (M-I) | 2.00 | 1.00 | 1.50 | 0.500 |

Example compositions 27 to 30 were filled into an aluminum spray can, optionally coated with epoxy-phenol lacquer, in a weight ratio of propellant (butane/propane/isobutane mixture) to suspension of 80:20, 85:15, 60:40, and 90:10, respectively.

Cosmetic antiperspirant agents according to the invention (quantities are expressed in % by weight, based on the total weight of the propellant-free composition)

| | 31 | 32 | 33 |
|---|---|---|---|
| Aluminum chlorohydrate (ACH) | 33.0 | 33.0 | 33.0 |
| C₁₀-C₁₃ isoalkanes | 8.90 | 8.90 | 8.90 |
| Dow Corning ES-5227 DM | 1.40 | 1.40 | 1.40 |
| Isoceteth-20 | 0.500 | 0.500 | 0.500 |
| Dimethicone | 4.20 | 4.20 | 4.20 |
| Isopropyl myristate | 9.00 | 9.00 | 9.00 |
| 1,2-Propanediol | 7.00 | 25.0 | 25.0 |
| Phenoxyethanol | 0.500 | 0.500 | 0.500 |
| Fragrance | 2.50 | 2.50 | 2.50 |
| Compound of formula (M-I) | 2.00 | 0.500 | 1.50 |
| L-Menthol | 0.400 | 0.300 | — |
| trans-Anethol | — | 0.300 | — |
| Eucalyptol | — | 0.300 | — |
| Water | ad 100 | ad 100 | ad 100 |

Example compositions 31 to 33 were filled into an aluminum spray can, optionally coated with epoxy-phenol lacquer, in a weight ratio of propellant (butane/propane/isobutane mixture) to W/O emulsion of 80:20, 85:15, 60:40, and 90:10, respectively.

Cosmetic antiperspirant agents according to the invention in the form of 0/W emulsions (quantities are expressed in % by weight)

| | 34 | 35 | 36 |
|---|---|---|---|
| Aluminum chlorohydrate (ACH) | 13.0 | 13.0 | 13.0 |
| Potassium aluminum sulfate, | 1.50 | 1.50 | 1.50 |
| KAl(SO₄)₂•12 H₂O | | | |
| Talc | 1.0 | — | — |
| Bentonite | — | 1.00 | — |
| Hectorite | — | — | 5.00 |
| Brij S 2 | 2.50 | 2.50 | 2.50 |
| Brij S 721 | 1.50 | 1.50 | 1.50 |
| Fragrance | 1.10 | 1.10 | 1.10 |
| Arlamol E | 0.500 | 0.500 | 0.500 |
| Bisabolol | 0.100 | 0.100 | 0.100 |
| Dry Flo PC | 0.100 | 0.100 | 0.100 |
| Compound of formula (M-I) | 2.00 | 3.00 | 1.00 |
| Dow Corning 2501 Cosmetic Wax | 0.100 | 0.100 | 0.100 |
| Tocopheryl acetate | 0.100 | 0.100 | 0.100 |
| Water | ad 100 | ad 100 | ad 100 |

Cosmetic antiperspirant agents according to the invention (quantities are expressed in % by weight, based on the total weight of the propellant-free composition)

| | 37 | 38 | 39 | 40 |
|---|---|---|---|---|
| Aluminum chlorohydrate (ACH) | 33.0 | 33.0 | 33.0 | 33.0 |
| Cyclomethicone | 12.0 | 9.40 | — | — |
| C₁₀-C₁₃ isoalkanes | — | — | 9.40 | 8.90 |
| Dow Corning ES-5227 DM | — | 1.40 | 1.40 | 1.40 |
| Abil EM 90 | 3.00 | — | — | — |
| Brij IC 20 | — | — | — | 0.500 |
| Dimethicone | — | 4.20 | 4.20 | 4.20 |
| Isopropyl myristate | 9.00 | 9.00 | 9.00 | 9.00 |
| Compound of formula (M-I) | 2.50 | 1.00 | 3.00 | 0.500 |
| 1,2-Propanediol | 7.00 | 7.00 | 7.00 | 7.00 |
| Phenoxyethanol | 0.500 | 0.500 | 0.500 | 0.500 |
| Fragrance | 2.50 | 2.50 | 2.50 | 2.50 |
| Water | ad 100 | ad 100 | ad 100 | ad 100 |

Example compositions 37 to 40 were filled into an aluminum spray can, optionally coated with epoxy-phenol lacquer, in a weight ratio of propellant (butane/propane/isobutane mixture) to W/O emulsion of 80:20, 85:15, 60:40, and 90:10, respectively.

Cosmetic antiperspirant agents according to the invention in the form of W/O emulsions (quantities are expressed in % by weight)

| | 41 | 42 | 43 | 44 |
|---|---|---|---|---|
| 50% aluminum chlorohydrate | 62.5 | 62.5 | 60.0 | 58.0 |
| (Locron L) | | | | |
| Propylene glycol | 5.00 | 5.00 | 7.50 | 9.50 |
| C₁₂-C₁₅ alkyl benzoate | 8.04 | 8.04 | 8.04 | 8.04 |
| Dimethicone 2 cst | 6.43 | 6.43 | 6.43 | 6.43 |
| Dimethicone 5 cst | 1.57 | 1.57 | 1.57 | 1.57 |
| Polyethylene | 10.2 | 11.7 | 9.70 | 12.2 |
| Abil EM 90 | 0.998 | 0.998 | 0.998 | 0.998 |
| Abil EM 97 | 1.22 | 1.22 | 1.22 | 1.22 |
| Compound of formula (M-I) | 2.50 | 1.00 | 3.00 | 0.500 |
| Synthetic wax | 0.100 | 0.100 | 0.100 | 0.100 |
| Fragrance | 1.50 | 1.50 | 1.20 | 1.50 |

The following commercial products were used:

| | | Supplier/ |
|---|---|---|
| Commercial product | INCI | manufacturer |
| Abil EM 90 | Cetyl PEG/PPG-10/1 | Evonik |
| | Dimethicone | |
| Abil EM 97 | Bis-PEG/PPG-14/14 | Evonik |
| | Dimethicone, | |
| | Cyclomethicone | |
| Abil K 4 | Cyclomethicone | Goldschmidt |
| Arlamol E | PPG-15 Stearyl Ether | Croda |
| Bentone 38 V CG | Disteardimonium Hectorite | Elementis |
| | | Specialities |
| Brij IC 20 | Isoceteth-20 | Croda |
| Brij S 2 | Steareth-2 | Croda |
| Brij S 721 | Steareth-21 | Croda |
| Cutina ® CP | Cetyl Palmitate | BASF |
| Cutina ® FS45 | Palmitic Acid, Stearic Acid | BASF |
| Cutina ® HR | Hydrogenated Castor Oil | BASF |
| Dow Corning ® | Cyclopentasiloxane | Dow Corning |
| 245 | | |
| Dow Corning ® | Bis-PEG-18 Methyl | Dow Corning |
| 2501 | Ether Dimethyl Silane | |
| Dow Corning | Dimethicone, PEG/PPG-18/18 | Dow Corning |
| ES-5227 DM | Dimethicone in a 3:1 weight | |
| | ratio | |
| Dry Flo PC | Aluminum Starch | National |
| | Octenyl Succinate | Starch |
| Eumulgin ® B1 | Ceteareth-12 | BASF |
| Eumulgin ® B2 | Ceteareth-20 | BASF |
| Eumulgin ® B3 | Ceteareth-30 | BASF |
| Kesterwachs K62 | Cetearyl Behenate | Koster Keunen |
| Finsolv TN | C12-15 Alkyl Benzoate | Innospec |
| Locron L | Aluminum Chlorohydrate | Clariant |
| (AS = 50%) | | |
| Mergital ® CS 11 | Ceteareth-11 | BASF |
| Novata ® AB | Cocoglycerides | BASF |
| | (melting point 30-32° C.) | |
| Plantaren ® 1200 | Lauryl Glucoside, approx. | BASF |
| | 50% AS | |
| Plantaren ® 2000 | Decyl Glucoside, approx. | BASF |
| | 50% AS | |
| Sensiva ® SC 50 | 2-Ethylhexyl Glycerin Ether | Schülke & Mayr |

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic antiperspirant agent, comprising:
a) at least one substance selected from the group consisting of cosmetic oils which are liquid at 20° C. and 1013 hPa, odorants, and waxes;
b) at least one antiperspirant aluminum salt in a total quantity of 1 to 80% by weight, based on the total weight of the cosmetic antiperspirant agent; and
c) at least one compound of formula (M-I) 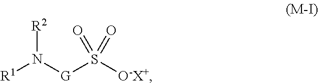
where
G stands for a chemical bond or a linear or branched alkylene group having 1 to 6 carbon atoms;
R¹ and R² independently stand for hydrogen or a linear or branched, saturated or unsaturated alkylene group having 1 to 10 carbon atoms, or R¹ and R² form a cyclic group having 5 to 7 carbon atoms and at least one heteroatom selected from the group of N, O, and S; and
X⁺ stands for H⁺, Li⁻, Na⁺, K⁺, NH₄⁺, ½Mg²⁺, ½Ca²⁺, ½Mn²⁺, ½Zn²⁺, ⅓Al³⁻, ¼Zr⁴⁺, or at least one antiperspirant aluminum salt.

2. The cosmetic antiperspirant agent according to claim 1, wherein the at least one substance (a) is an odorant and comprises 0.00001 to 10% by weight based on the total weight of the cosmetic antiperspirant agent.

3. The cosmetic antiperspirant agent according to claim 1, wherein the at least one substance (a) is a wax and comprises 0.01 to 20% by weight based on the total weight of the cosmetic antiperspirant agent.

4. The cosmetic antiperspirant agent according claim 1, wherein the at least one substance (a) is a cosmetic oil which is liquid at 20° C. and 1013 hPa, said cosmetic oil being selected from the group consisting of volatile cyclic silicone oils, volatile nonsilicone oils, nonvolatile silicone oils, nonvolatile nonsilicone oils, and mixtures thereof.

5. The cosmetic antiperspirant agent of claim 4, wherein the cosmetic oil includes a volatile cyclic silicone oils selected from the group consisting of cyclotrisiloxane, cyclotetrasiloxane, cyclopentasiloxane, and cyclohexasiloxane, and linear silicone oils having 2 to 10 siloxane units.

6. The cosmetic antiperspirant agent of claim 5, wherein the linear silicone oils having 2 of 10 siloxane units are selected from the group consisting of hexamethyldisiloxane, octamethyltrisiloxane, and decamethyltetrasiloxane.

7. The cosmetic antiperspirant agent of claim 4, wherein the cosmetic oil includes a volatile nonsilicone oils selected from the group consisting of liquid paraffin oils and isoparaffin oils.

8. The cosmetic antiperspirant agent of claim 7, wherein the cosmetic oil is selected from the group consisting of isodecane, isoundecane, isododecane, isotridecane, isotetradecane, isopentadecane, isohexadecane, and isoeicosane.

9. The cosmetic antiperspirant agent of claim 4, wherein the cosmetic oil is a high molecular linear polyalkylsiloxane.

10. The cosmetic antiperspirant agent of claim 4, wherein the cosmetic oil is selected from the group consisting of esters of linear or branched, saturated or unsaturated C₂₋₃₀ fatty alcohols with linear or branched, saturated or unsaturated C₂₋₃₀ fatty acids, which may be hydroxylated, the C₈-C₂₂ fatty alcohol esters of monovalent or polyvalent C₂-C₇ hydroxycarboxylic acids, the triethyl citrates, the branched saturated or unsaturated C₆₋₃₀ fatty alcohols, the mono-, di-, and triglycerides of linear or branched, saturated or unsaturated, optionally hydroxylated C₈₋₃₀ fatty acids, the dicarboxylic acid esters of linear or branched C₂-C₁₀ alkanols, the addition products of ethylene oxide and/or propylene oxide with monohydric or polyhydric C₃₋₂₂ alkanols, which may optionally be esterified, the symmetrical, asymmetrical, or cyclic esters of carbonic acid with fatty alcohols, the esters of dimers of unsaturated C₁₂₋₂₂ fatty acids with monohydric linear, branched, and cyclic C₂₋₁₈ alkanols or C₂₋₆ alkanols, the benzoic acid esters of linear or branched C₈₋₂₂ alkanols, the synthetic hydrocarbons, the alicyclic hydrocarbons.

11. The cosmetic antiperspirant agent according to claim 1, wherein the antiperspirant aluminum salt comprises 2 to 40% by weight of the cosmetic antiperspirant agent.

12. The cosmetic antiperspirant agent according to claim 1, wherein antiperspirant aluminum salt is selected from the group of water-soluble astringent inorganic salts of aluminum, water-soluble astringent organic salts of aluminum, water-soluble astringent inorganic aluminum-zirconium salts, water-soluble astringent organic aluminum-zirconium salts, and mixtures thereof.

13. The cosmetic antiperspirant agent according to claim 1, wherein the antiperspirant is selected from the group consisting of aluminum chlorohydrate, aluminum sesquichlorohydrate, aluminum dichlorohydrate, aluminum hydroxide, potassium aluminum sulfate, aluminum bromohydrate, aluminum chloride, aluminum sulfate, aluminum chlorohydrex propylene glycol, aluminum chlorohydrex polyethylene glycol, aluminum-propylene glycol complexes, aluminum sesquichlorohydrex propylene glycol, aluminum sesquichlorohydrex polyethylene glycol, aluminum dichlorohydrex propylene glycol, aluminum dichlorohydrex polyethylene glycol, aluminum undecylenoyl collagen amino acid, sodium aluminum lactate, sodium aluminum chlorohydroxy lactate, aluminum lipoamino acids, aluminum lactate, aluminum chlorohydroxy allantoinate, sodium aluminum chlorohydroxy lactate, aluminum zirconium trichlorohydrate, aluminum zirconium tetrachlorohydrate, aluminum zirconium pentachlorohydrate, aluminum zirconium octachlorohydrate, aluminum-zirconium/propylene glycol complexes, aluminum zirconium trichlorohydrex glycine, aluminum zirconium tetrachlorohydrex glycine, aluminum zirconium pentachlorohydrex glycine, aluminum zirconium octachlorohydrex glycine, and mixtures thereof.

14. The cosmetic antiperspirant agent according to claim 1, wherein the compound of formula (M-I) comprises 0.05 to 8% by weight of the cosmetic antiperspirant agent.

15. The cosmetic antiperspirant agent according to claim 1, wherein the cosmetic antiperspirant agent comprises at least one compound of formula (M-Ia) and/or (M-Ib) 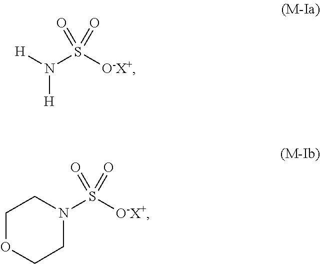
where
X⁺ stands for H⁺, Li⁺, Na⁺, K⁺, NH₄⁺, ½Mg²⁺, ½Ca²⁻, ½Mn²⁻, ½Zn²⁺, ⅓Al³⁺, ¼Zr⁴⁺, or at least one antiperspirant aluminum salt.

16. The cosmetic antiperspirant agent according to claim 1, wherein the cosmetic antiperspirant agent has a weight ratio of the antiperspirant aluminum salt to the compound of formula (M-I) of 40:1 to 19:1.

17. The cosmetic antiperspirant agent according to claim 1, wherein the cosmetic antiperspirant agent has a weight ratio of the antiperspirant aluminum salt to the compound of formula (M-I) of 10:1 to 13:1.

18. The cosmetic antiperspirant agent according to claim 1, wherein the cosmetic antiperspirant agent includes free water in a total quantity of 15 to 96% by weight based on the total weight of the cosmetic antiperspirant agent.

19. The cosmetic antiperspirant agent according to claim 1, wherein the cosmetic antiperspirant agent includes ethanol in a total quantity of 1 to 50% by weight based on the total weight of the cosmetic antiperspirant agent.

20. A method of activating and/or stabilizing at least one antiperspirant aluminum salt, comprising:
combining at least one antiperspirant aluminum salt with at least one compound of formula (M-I) 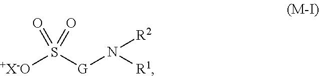
where
G stands for a direct bond or a linear or branched alkylene group having 1 to 6 carbon atoms,
R¹ and R² independently stand for hydrogen or a linear or branched, saturated or unsaturated alkylene group having 1 to 10 carbon atoms, or R¹ and R² form a cyclic group having 5 to 7 carbon atoms and at least one heteroatom selected from the group of N, O, and S, and
X⁺ stands for H⁺, Li⁺, Na⁺, K⁺, NH₄⁺, ½Mg²⁺, ½Ca²⁺, ½Mn²⁺, ½Zn²⁺, ⅓Al³⁻, ¼Zr⁴⁺, or at least one antiperspirant aluminum salt.
